(19) United States
(12) Patent Application Publication
(10) Pub. No.: US 2006/0195035 A1
(43) Pub. Date: Aug. 31, 2006

(54) Non-invasive radial artery blood pressure waveform measuring apparatus system and uses thereof

(76) Inventor: Dehchuan Sun, Taipei City (TW)

Correspondence Address: BRUCE H. TROXELL, SUITE 1404, 5205 LEESBURG PIKE, FALLS CHURCH, VA, 22041, US

(21) Appl. No.: 11/066,291

(22) Filed: Feb. 28, 2005

(51) Int. Cl. A61B 5/02

(52) U.S. Cl. 600/503; 600/485

(57) ABSTRACT

The invention relates to a non-invasive apparatus system for measuring radial artery blood pressure waveform, and its uses in heart rate variability measurement, autonomic nervous system measurement, personal identification, respiratory cycle and cough monitoring, home quarantine, and hospital quarantine thereof.

### BACKGROUND OF THE INVENTION

1. Field of the Invention

The present invention relates to a non-invasive apparatus system for measuring radial artery blood pressure (BP) waveform by applying an apiezoelectric sensor, particularly to a measuring apparatus system, which can be used in heart rate variability (HRV) measurement, autonomic nervous system. (ANS) measurement, personal identification, respirotary cycle and cough monitoring, home quarantine, and hospital quarantine thereof.

2. Description of the Prior Art

Heart rate (HR) and blood pressure (BP) (systolic blood pressure (SBP) and diastolic blood pressure (DBP)) are two important physiological parameters, which can be measured through a conventional electronic wrist or wrist blood pressure (BP) apparatus. Such apparatus has become one of the most necessary medical appliances due to easy uses and reasonable costs. BP waveform, another crucial hysiological parameter, however, may not be measured through any easily operating and accurate apparatus system yet.

The electronic BP measuring apparatus currently available in the market may only measure and show SBP (the maximum value of BP waves) and DBP (the minimum value of BP waves).

A conventional electronic wrist blood pressure (BP) apparatus usually comprises a pump for pumping airs, an air escape valve, a barometer, an air duct, a wristlet with an included air bag, a circuit module, and a casing. When measuring BP, the air pump and air escape valve pressurize and decompress said air bag, meanwhile, wrist radial artery BP is transmitted to the barometer through an air bag. Then, software/hardware installed on a circuit magnifies and filters the pulse wave signals of said barometer to estimate HR, SBP, and DBP.

Physiologically, besides HR, SBP, and DBP, precise radial artery BP waveform (its clinical applications will be detailed later) is also a crucial physiological parameter. Even though the BP measuring technique mentioned above can be conducted by recording radial artery BP waveform with a barometer, due to the factors such as material characteristic of said air bag and geometric shape, original form of BP waves may not be delivered to said air bag. Moreover, BP waveform is dampened and weakened when it is delivered from said air bag, through air duct, to barometer, which results in the loss of sensitivity and accuracy of measured BP waveform. In order to ensure more accurate radial artery BP waveforms, the sphygmography currently available in the market has been installed with a circular-shaped pressure sensor having about 5-mm diameter. When performing measurements, the circular-shaped pressure sensor is attached onto the wrist radial artery with an adhesive tap or rubber band at first, then the signal wire is connected with a circuit board or computer. Generally, the circular pressure sensor is made of Resistor-type conductive materials, and its principle is similar to a Strain Gauge's, including components such as magnification circuits, temperature effect compensation, and linear processing. When resistor-type materials are under pressure, the variable of resistance, current, or voltage is proporational to pressure value, by which the pressure value can be recorded. The technique stated above can actually measure the specifics and original form of radial artery BP waveforms. However, it fails to accurately control the pressure occurred when fixing a circular pressure sensor onto the wrist.

It can be found through a test that the pressure occurred when fixing the circular pressure sensor onto the wrist is crucial to the measurement of BP waveforms. If the pressure is too light (i.e. only fixing the sensor with an adhesive tape or rubber band as stated above), under some circumstances such as fleshy wrist, soundless radial artery, or thready Pulse, the pressure sensor may not obtain clear BP waveform signals. If the pressure is too heavy, radial artery blood stream is greatly obstructed, which results in distortion of measured BP waveform. Moreover, due to factors such as the wrist shape and size and deep position of radial artery, the circular-shaped strain gauge may not find correct artery position unless a trained doctor or nursing staff feels the pulse with fingers to find correct artery and fasten the circular-shaped strain gauge thereon. Such operation is not easy and convenient. That is one reason why the sphygmography or similar apparatus could not be used as home medical appliances like a digital BP monitor.

Moreover, there have been proposed a variety of conventional personal identification techniques, including facial image analysis, voice recognition, fingerprint recognition, blood type, eyes, hair, and handwriting analysis, and advanced DNA cell identification analysis. The present invention found from a test that each artery BP waveform is unique due to the difference of individual's heart size and shape, myocardium structure, and arterial tree structure, and can be regarded as personal identification characteristics. Although artery BP waveforms may vary due to factors such as emotions and environments (i.e. HR accelerates or BP increases when feeling nervus or angry), only if the normalization of BP waveform to HR and BP value, waveform characteristics will become stable as individualized characteristics.

Currently, the typical apparatus for measuring heart rate variability (HRV) and autonomic nervous system (ANS) is electrocardiograph (ECG or EKG) machine. HRV refers to the heartbeat rate (or HR). Besides, Homeostasis remains about 60-90 times per minute, some regular or irregular wave motions have been hided therein. When using an electrocardiograph (ECG) machine to measure HR and its variability, electrodes need to be stuck onto a patient's hand and foot (and thoracic cavity) in order to measure periodical ECG signal, and then estimate the peak-to-peak interval (i.e. R-R interval, R peak is the highest peak of ECG wave) of measured ECG wave, Theough the peak-to-peak interval sequence, each parameter of HR and HRV can be estimated further. For example, the average of Peak-to-Peak interval sequence is heart period; the reciprocal of heart period is heart rate (HR); the standard deviation of peak-to-peak interval sequence is heart rate variability (HRV); such peak-to-peak interval sequence data can be transformed to a spectrum through the fast fourier transform (FFT). Through the spectrum analysis, total power of HRV can be divided into two components—high frequency (HF, 0.15-0.4 Hz) component and low frequency (LF, 0.04-0.15 Hz) component. Through animal and human body experiments, De Boer et al. (Hemodynamic Fluctuations and Baroreflex Sensitivity in Humans: A Beat-to-Beat Model.; American Journal of Physiology; 253: H680-H689; 1987) verified that HRV (that is, standard deviation of peak-to-peak interval sequnece), total power represents autonomic nervous activity, low frequency represents sympathetic nervus system (ANS), high frequency represents parasympathetic nervus system, and the ratio of low frequency to high frequency (LF/HF) represents automatic nurve balance. Since autonomic nervous system controls various conscious and unconscious body activities, such as HR, BP, blood suger, sleep, perspiration, bronchiectasis, and so on, there is a need to provide a user-friendly and low-price automatic nurver monitor for medical use. Currently, the field of medical science uses the electrocardiograph (ECG) machine to measure HRV and ANS capability, but such operation is not only complicated (i.e. requiring large-scale apparatus and specialized software, pasting many electrodes, testee's action is restricted, etc.), but also high-cost (initial apparatus and software cost, and follow-out training and electrode cost).

Upon inspiration, signals from respiratory center in brain spillovers to vasomotor center. Through autonomic Nerve (sympathetic nerve and para-sympathetic nerve) reflex, spillover signal makes HR and systole increase and decrease regularly in accordance with respirotary cycle, therefore, artery BP waveform can be accurately recorded, HR variation is further estimated and analyzed, and then respirotary frequency and waveform is detected. In addition, the peak of arterial pressure (corresponding to the specific point of SBP) and trough (corresponding to the specific point of DBP) also rise and fall in accordance with the respirotary cycle (also called as arterial respiratory waves in medical terms). In addition to the factors such as signal spillover of respiratory center, arterial respiratory waves are also derived from another two psysiological functions, as follows: (1) diaphragm descends upon inspiration to bring negative pressure, while the amount of blood flew from blood vesels to heart decreass, leading to the decrease of Cardiac Output and immediate drop of BP; (2) blood vessels of thoracic cavity has changes in pressure due to ascending and descending of the diaphragm; through Baroreceptor Reflex, such changes make arterial pressure rise and fall in accordance with respirotary frequency (for details on the physiological phenomenon and principle mentioned above, refer to “Textbook of Medical Physiology”, Authored by Arthur C. Cuyton, Eighth Edition, W. B. Saunders Company, ISBN 0-7216-3087-1, 1991, Chapter 13). As understood from above, through precise recording of artery BP waveform, respirotary frequency and waveform can be obtained through analyzing the rising and falling of the peak or trough.

Moreover, infectious disease prevention and medical care is a focal point in the medical system. Take the Severe Acute Respiratory Syndrome (SARS) inflicted throughout Asia in recent years for example, since the routes of SARS infection are mainly person-to-person air-borne infection, the most effective strategy of SARS prevention is isolating the patients with others. As regards other infectious diseases, regardless of diseases inflected through airs (i.e. tuberculosis) or blood and body fluid (i.e. AIDS), one of the most important issues in the field of health care is how to decrease close contact between patients (or suspected patients) and medical personnel or family. Furthermore, as regards hospital quarantine and monitoring, the current standard treatment procedures in the hospital include: medical personnel get into isolation wards daily to measure patients' body temperature, heartbeat, and BP several times(i.e. four times in one day) and observe the patients' symptoms (i.e. dyspnea and cough). The execution of this procedure often makes the medical personnel subjected to be inflected. Accordingly, if the isolated patient's physiological signals can be automatically delivered from isolation wards to nursing station, the probability of infection through person-to-person contact can be reduced. In addition, major symptoms of common infectious diseases include favor, palpitations, tachypnoea, cough, sneeze, abnormal BP, and so on. When the non-invasive radial artery BP waveform measuring technique, conventional body temperature measuring technique, as well as conventional wireless or wired transmission techniques in the present invention are integrated, as long as isolated patients carry the wrist physiological monitor of the present invention (will be detailed latter), their physiological signals (including body temperature, HR, BP, respirotary waveform, and cough) will be delivered out of a ward, so that the purpose of reducing close contact can be achieved.

As regards home quarantine and monitoring, in view of the SARS prevention experiences recently, the biggest loophole in epidemic prevention is that those who are subject to home quarantine leaves home without permission. Another technical demand of home quarantine is obtaining isolated people's physiological signals regularly (i.e. everyday) to control overall epidemic situation. Currently, health organizations in many countries use the approach of sending related personnel to investigate isolated people. However, such measure not only requires a lot of human resources, but also makes infection through close contact to occur easily. Through integrating the apparatus and methods in the present invention, including: (1) non-invasive wrist BP waveform measuring technique, (2) personal identification technique, (3) respiratory waveform technique, and (4) cough monitoring technique; and conventional (1) blood temperature measurement technique,(2) wireless or wired transmission technique, and (3) BP measurement technique, the physiological signals (such as body temperature, HP, BP, cough, etc.) of those who are subject to home quarantine can be regularly sent to hospitals or the health authority. This not only prevents those who are subject to home quarantine from leaving home without permission (or from being substituted by others), but also estimates numbers of the sick and their location, so that the epidemic can be controlled.

As described above, BP waveform rises and falls periodically during normal breathing. However, if a person coughs or sneezes suddenly, his diaphragm and thoracic cavity vibreates rapidly, which cause irregular change of BP waveform quickly. When cough or sneeze stops, the BP waveform resumes normal condition. Accordingly, through the analysis of personal BP waveform baseline and abrupt change, the symptoms (such as cough or sneeze) a testee may have can be detected.

R.O.C. patent 363404 disclosed using an ECG converter (which contains electrodes) for analyzing HRV to measre electric signals occurred due to systole, and estimating HRV through the fourier transform and spectrum analysis. However, the purpose of such invention is to provide a newest ECG converter for analyzing HRV. It features the design the new hardware and software and apparatus system.

R.O.C. patent 176323 disclosed using a non-invasive autonomic nervous system monitoring apparatus system to monitor the autonomic nervous system side effect of patients who take medicine, as well as aging degree or treatment effects.

In the prior art described above, the previous invention did not disclose any new non-invasive piezoelectric sensor for accurately measuring wrist radial artery BP waveform as shown in the present invention. Moreover, the invention did not disclose any new non-invasive piezoelectric sensor for uses in HRV measurement, ANS measurement, personal idntification, respirotary cycle and cough monitoring, home quarantine, and hospital quarantine, either.

### SUMMARY OF THE INVENTION

Accordingly, a primary purpose of the present invention is to overcome above-mentioned technical and operating difficulties to develop a set of non-invasive, user-friendly apparatus system for accurately measuring radial artery blood pressure (BP) waveform.

Another purpose of the present invention is to measure physiological mean blood pressure (PMBP) by applying the non-invasive BP waveform technique, which can accurately measure BP waveform and estimate PMBP through the integral value of BP waveform—time chart, and SBP and DBP measured by a conventional electronic BP measuring apparatus.

Another purpose of the present invention is to monitor heart rate variability (HRV) and autonomic nervous system (ANS) by applying the present non-invasive, user-friendly, low cost, and highly accurate radial artery BP waveform measuring apparatus system.

Another purpose of the present invention is to use the present non-invsive BP waveform measuring apparatus system for uses in personal identification.

Another purpose of the present invention is to use the present non-invsive lood pressure waveform measuring technique to achieve the objective of monitoring physiological parameter such as respirotary frequency and waveform.

Another purpose of the present invention is to develop a set of non-invasive apparatus system by which hospital quarantine and monitoring of infectious diseases can be conducted without close person-to-person contact.

Another purpose of the present invention is to use the present non-invasive BP waveform measuring apparatus system to achieve the object of cough or sneeze monitoring.

A further purpose of the present invention is to develop a set of non-invasive apparatus system for uses in monitoring home quarantine patients.

The embodiments taken in conjunction with the accompanying drawings are described below on order to achieve the technical purposes as mentioned above.

### BRIEF DESCRIPTION OF THE DRAWINGS

FIG. 1 is a block diagram of the present apparatus system;

FIG. 2 is a view showing an embodiment of using a piezoelectric sensor in the present invention;

FIG. 3a is view showing the relationship between air pressure of said air bag and time according to the present invention;

FIG. 3b is a view showing the relationship between blood pressure waveform and air pressure according to the present invention;

FIG. 3c is view showing the wrist radial artery BP waveform according to the present invention;

FIG. 4 is a view showing another embodiment of using a piezoelectric sensor in the present invention;

FIG. 5 is a view showing another embodiment of using a piezoelectric sensor in the present invention;

FIG. 6 is a view showing a further embodiment of using a piezoelectric sensor in the present invention;

FIG. 7 is a flow diagram of the operating software in the present invention;

FIG. 8 is an illustration showing how to measure PMBP through using the blood pressure (BP) waveform according to the present invention;

FIG. 9a is an illustration showing the radial artery blood pressure (BP) waveform measured in the present invention;

FIG. 9b is another illustration showing the radial artery blood pressure (BP) waveform measured in the present invention;

FIG. 9c is another illustration showing the radial artery blood pressure (BP) waveform measured in the present invention;

FIG. 10 is a view showing the continuous blood pressure (BP) waveform measured in the present invention;

FIG. 11 is an illustration showing the respiratory waveform measured in the present invention;

FIG. 12 is another illustration showing the respiratory waveform measured in the present invention;

FIG. 13 is a view showing the use of blood pressure (BP) waveform to monitor cough and sneeze according to the present invention;

FIG. 14 is a view showing the physiological signal monitoring apparatus system for uses in hospital quarantine monitoring;

FIG. 15 is a view showing the physiological signal monitoring apparatus system for uses in home quarantine monitoring;

FIG. 16 is an illustration showing another embodiment of the present invention;

FIG. 17 is a view showing the wrist radial artery blood pressure (BP) waveform measured in the present invention;

FIG. 18a is a view showing the first wrist radial artery blood pressure (BP) waveform measured in the present invention;

FIG. 18b is a view showing the second wrist radial artery blood pressure (BP) waveform measured in the present invention;

FIG. 19 is a view showing the third wrist radial artery blood pressure (BP) waveform measured in the present invention;

FIG. 20 is a view showing the fourth wrist radial artery blood pressure (BP) waveform measured in the present invention.

### DETAILED DESCRIPTION OF THE EMBODIMENTS

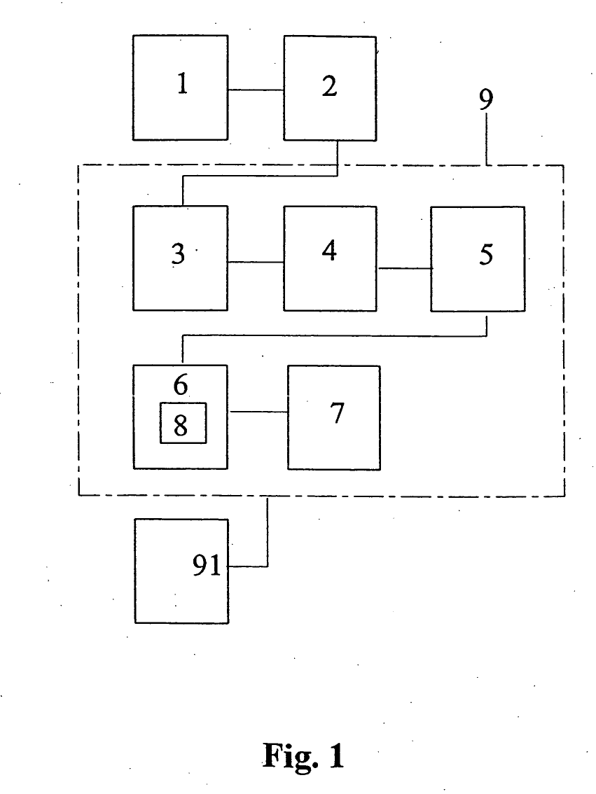

The non-invsive radial artery blood pressure (BP) waveform measuring apparatus system according to the present invention, as shown in FIG. 1, comprising:
(a) a piezoelectric sensor 1 for measuring wrist radial artery BP, which can continuously record and form electric waves on behave of BP waves;
(b) a wristlet 2 with an included air bag can be put on one's wrist, and pushes said piezoelectric sensor 1;
(c) components such as pump 3, air escape valve 4, air duct 5 that can fill or escape airs in said air bag;
(d) circuit module 6, comprising a central processing unit (CPU),memory, and barometer 7 connected with aur duct of said air bag;

(e) operating sorftware 8 installed in said circuit module, which can control air pressure of said air bag, finding out Optimal testing air pressure, measure radial artery BP waveform under Optimal testing air pressure, and filter, magnify, and analyze electric waves derived from piezoelectric sensor 1;
(f) power source 91; and
(g) a host 9 with a casing, which comprises the component (c) to (f).

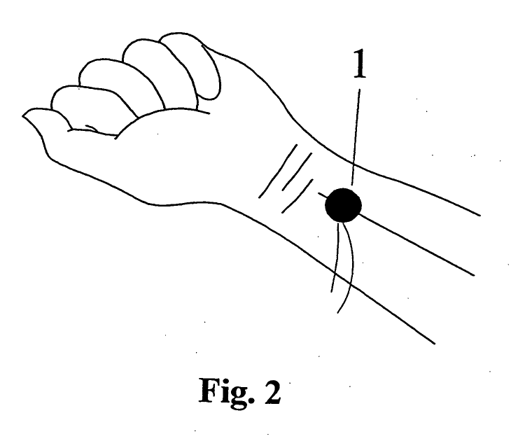

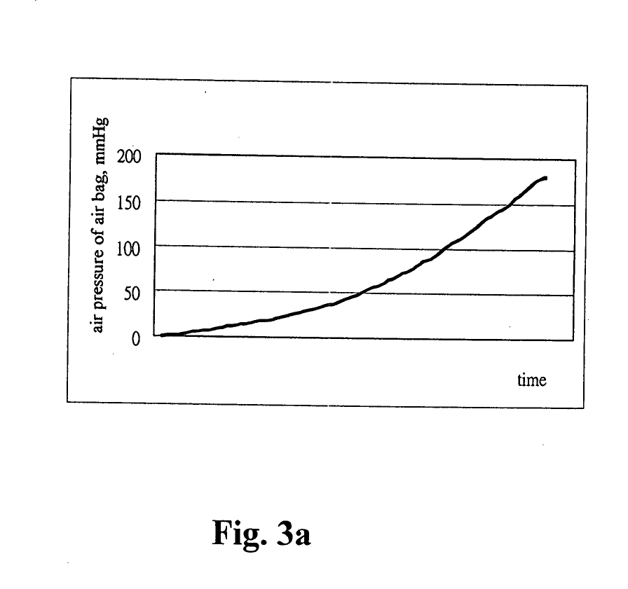

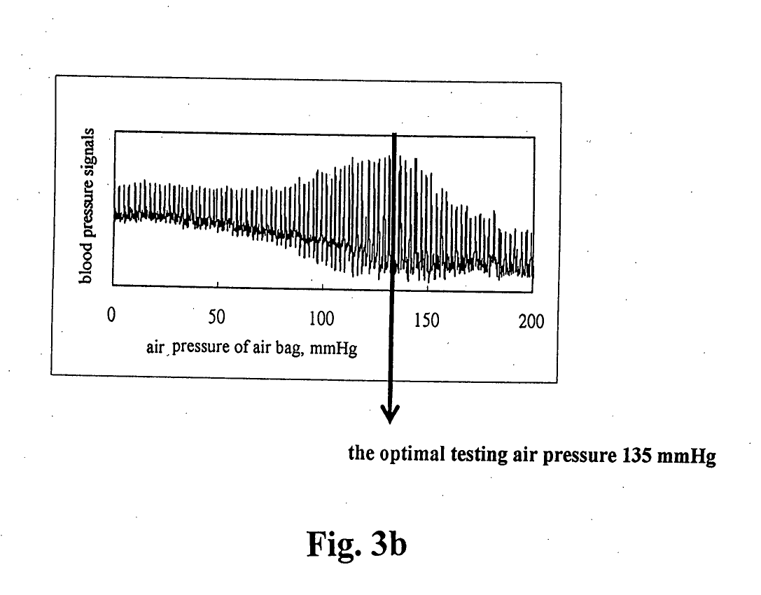

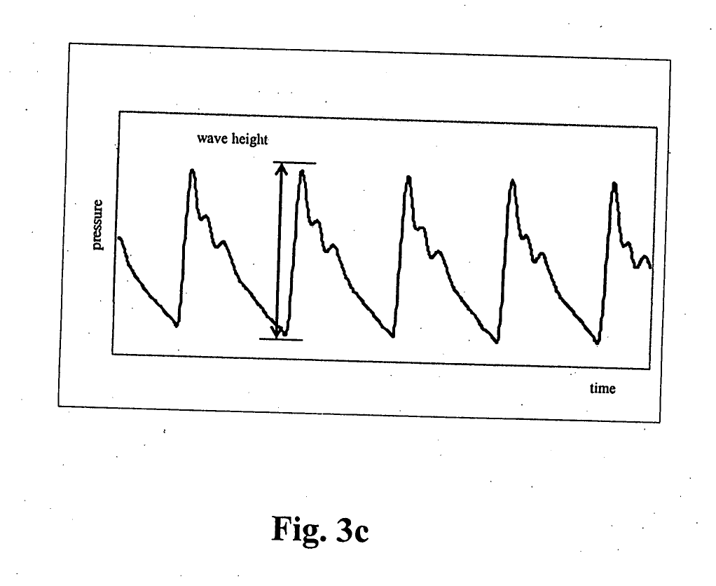

According to the present invention, the piezoelectric sensor 1 can be, but is not limited, a conventional ceramic Lead zirconate titanate (PZT) piezoelectric transducer, polyvinylidene fluoride (PVDF) piezoelectric transducer, strain gauge piezoelectric devices, or semi-conductor silicon piezoelectric device, and so on; said sensor 1 may stand alone from the wristlet or other hardware, but it can transmit electric wave signals to circuit module 6 through a connector. When testing is carried out, firstly, said sensor 1 is fastened above the wrist radial artery blood with an adhesive plaster or elastic bands (i.e. elastic or rubber bands) (as shown in FIG. 2), the wristlet installed with a host is then put on one's wrist, wherein the air bag in the wristlet exactly withholds said sensor. When said air bag is pressurized by a pump, it pressurizes the sensor; meanwhile, said sensor pressurizes radial artery and thus strengths the pulse wave signal obtained by the sensor (referring to FIG. 3a, 3b, 3c).

A focal point of the inventive technique is using the pump to increase pressure of the air bag by degrees from zero to a certain value (i.e. 200 mmHg, FIG. 3a), recording the BP pulse waves (FIG. 3b) obtained by a sensor, and computing the primary peak height of each pulse wave (in FIG. 3c). Generally, primary peak height increases during air filling and decrease then. When the primary peak height reaches the maximum (normally within 70-150 mmHg, FIG. 3b), the pressure of air bag is defined as Optimal testing air pressure according to the present invention. In this case, the sensor's BP waveform signal is strongest ever, that is, signal-to-noise ratio is highest. When the pressure value of air bag is lower than this value, inferior transmission of pressure between the piezoelectric sensor and radial artery results in fading of pulse wave signals; when the pressure value of air bag is higher than this value, the overpress upon radial artery casued by wristlet, air bag, and sensor decreases artery blood stream and causes fading and distoration of pulse wave signals.

One of crucial procedure taken in the present invention is obtaining optimal testing air pressure (OTAP) through the above-mentioned air filling step before measuring radial artery BP waveform, pressurizing the air bag until (or close) such pressure value, and then monitoring the BP waveform. If the air filling procedure described above is changed to pressurize the air bag to a specific air pressure value (i.e. 200 mmHg) first, and then descrease the air pressire little by little, optimal testing air pressure (OTAP) can still be found during the period of air escape.

The shape and size of the piezoelectric sensor described above did not affect the implementation of the present invention. According to the present invention, the sensing body of said piezoelectric sensor may be a thin film with circular, square, or other geometric shape; its thickness ranges from 0.1 mm to 5 mm, and its diameter or side ranges from 1 mm to 100 mm. Perferably, the piezoelectric sensor is a circular thin film, in which its diameter is about 2 to 5 mm, and its thickness is about 0.1 to 3 mm. Its electric wave signals are transmitted to a circuit module through two positive/negative wires, and its power source (strain gauge piezoelectric devices or semi-conductor silicon piezoelectric device needs extra power source, while PZT or PVDF piezoelectric transducer do not need) are provided by the battery of circuit module or external power source through wires.

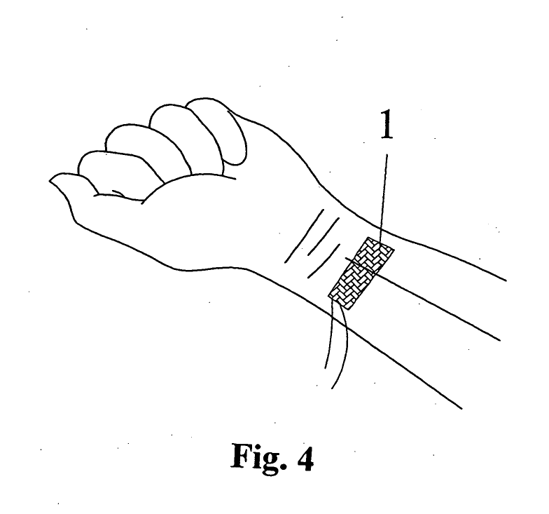

During the test, feel the pulse with fingers to find out the location of radial artery, and then fasten a piezoelectric sensor above the radial artery. According to another embodiment of the present invention as shown in FIG. 4, preferably, piezoelectric sensor 1 is a rectangle thin film, wherein its length (parallel to hand length) is about 1 to 30 mm, and its width (parallel to hand width) is about 15 to 60 mm. Other connection or supplied power characteristics are the same as ones described above. During the test, since the range covered by the sensor is large enough, the sensor can be fastened around the position of radial artery (instead of the accurate position of radial artery) to proceed subsequent tests.

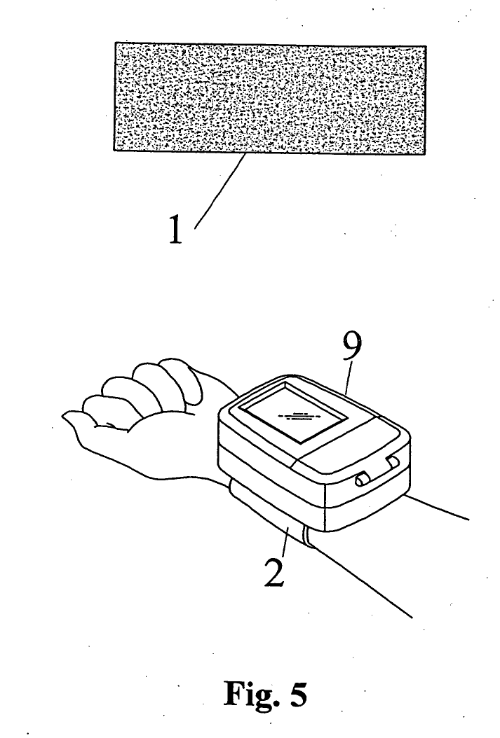

As shown in FIG. 5 in the present invention, another embodiment with regards to the piezoelectric sensor is installing said rectangle thin film inside the wristlet 2, wherein one of said thin film contacts with air bag in the wristlet, and another side contacts with outer cloth of the wristlet. When a test is conducted, the wristlet 2 with host 9 installed thereon only need to be put on one's wrist.

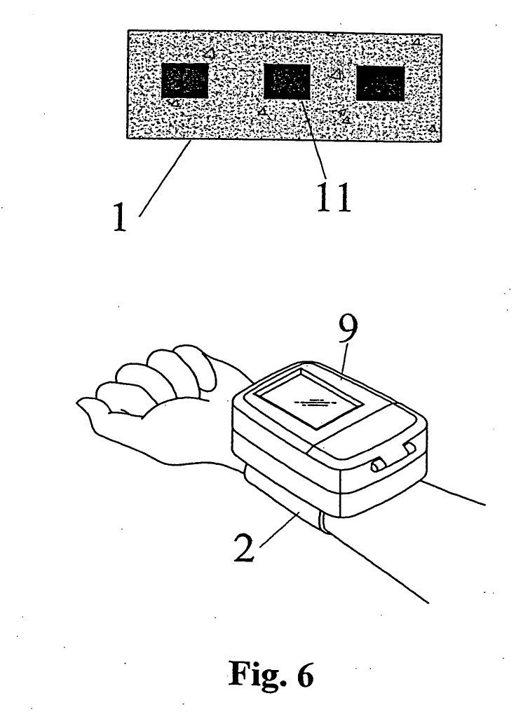

As shown in FIG. 6, the piezoelectric sensing module of piezoelectric sensor 1 may comprise a plurality (at least two) of piezoelectric devices 11, and is installed with a wristlet 2, wherein each device is a rectangle (or circular) piezoelectric thin film, wherein its side (or diameter) is 3 to 5 mm; the base of said sensing module is a soft printed circuit board, which is assembled according the following steps: aigning a plurality of piezoelectric devices 11 in one line parallel to hand width, and fastening them onto the soft printed circuit board, a slot (0.1 to 1 mm) is disposed between the device and to prevent the interference of adjacent electric waves.

Moreover, a multiplexer and its driver software are provided onto the circuit module of the present invention. Said soft printed circuit board transmits electric wave signals derived from sensing devices to the multiplexer through a wire or connector, and then the signals are connected to signal filter and amplification circuits. When measuring BP and pulse waves, the multiplexer gathers electric wave signals of each piezoelectric device on the sensing module. As compared with other devices, said device is located on or near radial arteries, and thus can obtain the strongest singals.

According to the present invention, a device having the strongest signal is selected as measuring device, while signals of other devices are no longer used. Other measuring procedures (i.e. optimal testing air pressure) are similar to the embodiments described above. At least two (properably 3 to 5) multiple devices described above are needed, so that the hand width ranged from 10 to 25 mm can be covered. Compared with other examples described above, the piezoelectric sensing module is preferably applied to various wrist sizes because soft printed circuit board is used as its base, which can be closely stuck upon the wrist surface.

As to the three cases concerning the piezoelectric sensor mentioned above, they have the merits of expanding the range of detecting pulse wave, omitting the step of searching for pulse, and suiting for different wrist size. Besides, the general public can measure by themselves instead of professed doctors and nurses.

According to the present invention, said wristlet can be cloth wristlet usually used in a digital wrist BP monitor, which includes an air bag whose size depends on the regulation on wrist BP measurement (its length (parallel to hand length) is about 60 to 90 mm, and its width (parallel to hand width) is about 80 to 150 mm. The plastic (or rubber) airproof air bag usually includes two gas pin with one connected to the barometer of circuit module and another connected to an air duct in connection with a pump and air escape valve. When the pump receives the program command of the present invention and starts pump up, the air escape valve closes temporarily and the pressure of air bag increases, wherein its value is monitored by a barometer; when the air escape valve receives program commands and starts to escape airs, said air escape valve opens, thus the pressure of air bag decreases. For wearing easily, the wristlet usually comprises a U-shaped or [symbol] -shaped plastic film, wherein the size of its opening is equal to the thickness of one's wrist, so that the wristlet with an included host can be put on one's wrist easily. The plastic film also comprises a protruding cramp, which is exposed outside the wristlet cloth, and its function lies in integrating with the casing of the inventive host to fasten the host upon the wristlet. In addition, referring to the embodiments as illustrated in FIGS. 4 and 5, the air bag may be added with a pocket to include the piezoelectric sensing device therein. When the air bag is filled with airs, it can accurately press the piezoelectric sensor; similarly, the pocket of said piezoelectric sensor can be fixed anywhere inside the wristlet (i.e. between wristlet cloth and air bag) to facilitate pressurization.

According to the present invention, wherein said pump is similar to the air pump used in conventional digital BP monitors, which accepts direct current (DC) to push and make blades rotate to pump airs. According to the present invention, the air escape valve may be similar to the electromagnetic valve On-Off switch used in a conventional digital BP monitor, which accepts program command to open or close air valve; said air escape valve may be an analog electromagnetic valve switch, wherein the degree of valve open depends on the voltage or current value, and such analog valve has better control over air escape speed than the On-Off switch (only close or open). According to the present invention, the base of circuit module may be a Printed Circuit Board, which is installed with a central processing unit (CPU), memory (i.e. Flash or RAM, etc.), barometer, signal filter device, signal magnification device, buzzer, Real-Time Clock, and other electric components. To facilitate display of the test process and results, circuit module may contain a LCD or LED display. Moreover, a Multi-Plexer can be installed onto the circuit module to conform to better circuit requirements as shown in FIG. 6 described above.

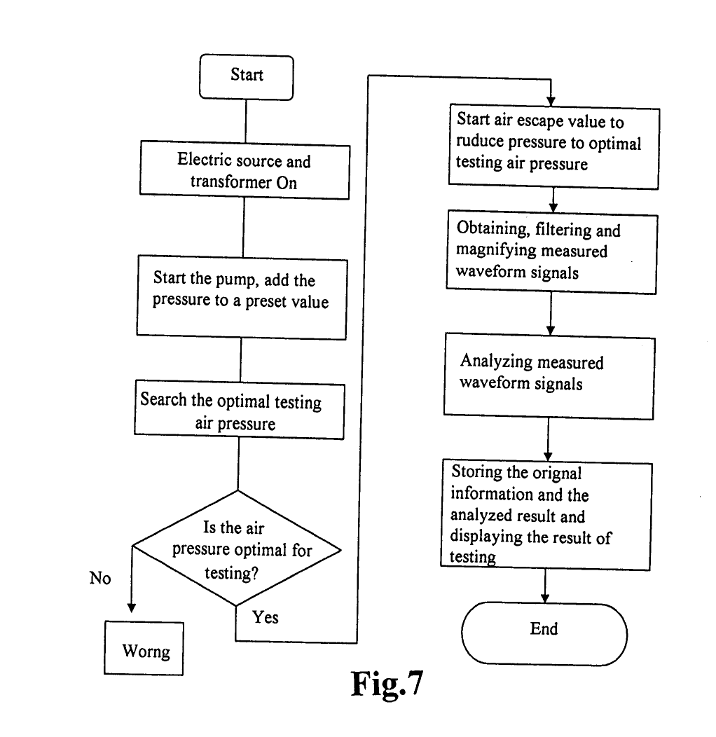

According to the present invention, the circuit module is installed with operating software, which can drive related hardware (such as pump, air escape valve, central processing unit (CPU), barometer, LCD displays, piezoelectric sensor, etc.) according to the flowchart in FIG. 7 to achieve the objectives as stated below:
(1) controlling air pressure in an air bag;
(2) finding out optimal testing air pressure;
(3) measuring radial artery BP waveform under the optimal testing air pressure;
(4) filtering and magnifying measured waveform signals;
(5) analyzing and computing measured waveform signals.

According to the present invention, wherein one of power sources can be typical dry cells (disposable), a lithium battery (rechargeable), a Ni-NM battery (rechargeable), or other kinds of battery. Further, the power source used in the present invention can be offered by conventional alternating current (AC, i.e. 110V or 220V). If alternating current (AC) is used, an AC/DC transformer can be added onto the circuit module of the present invention to provide power source for electric devices that only accepts direct current (DC).

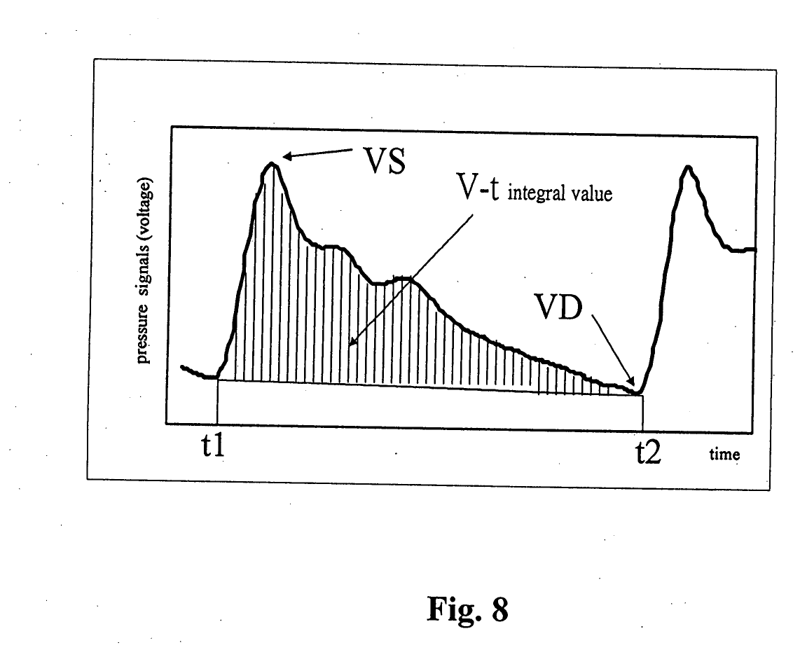

As to using the non-invasive BP waveform measuring technique according to the present invention, PMBP can be measured according to the following steps:
(a) measuring SBP and DBP through conventional digital BP monitor oscillometric method;
(b) accurately measuring a BP waveform according to the present invention, and drawing a X-Y plane diagram (FIG. 8), wherein Y axle represents relative voltage value V, X axle represents real time t;
(c) estimating V(mean) of BP waveform within (b) according to the following formula:
   V(mean)=V-t integral value/(t2-t1) (1)

Wherein V(mean) represents relative mean voltage, V-t integral value represents the integral value within t1 (the starting point of waveform) to t2 (the endpoint of waveform) domain in V-t waveform diagram, t2-t1 represents the time interval of individual waveform;
(d) PMBP is estimated according to the following formula:
   MBP=V(mean)*(SBP−DBP)/(VS−VD) (2)

Wherein PMBP represents physiological mean blood pressure, V(mean) represents mean voltage, SBP represents systolic blood pressure, DBP represents Diastolic blood pressure, VS represents the maximum value (peak) of BP waveforms, and VD represents the minimum value (trough) of BP waveform.

To obtain more representable and accurate PMBP, an embodiment of the present invention is collecting more than one BP waveform, estimating PMBP of each BP waveform, and then obtaining the mean of a number of PMBP according to the following formula:
PMBP(mean)=SUM(PMBP)/N (3)

Wherein PMBP(mean) represents the mean of physiological mean blood pressure (PMBP), SUM(PMBP) represents the sum of N PMBP, N represents the number of BP waveform. As to the digital BP oscillometric method stated above, refer to U.S. Pat. No. 4,860,760. Typical BP oscillometric method is summzrized as follows:
(a) pressurizing the air bag until a certain value (i.e. 200 mmHg);
(b) recording barometer's BP waveform during gradual air escape, estimating peak height, and drawing a peak height-time (X-Y) diagram;
(c) finding out the maximum value of waves height within air escape shown on the illustration;
(d) using the maximum value of the wave height as baseline, finding out the pressure corresponding to 50% of the maximum wave height in accordance with the pressure rising direction of X-axle, which represents the SBP;
(e) using the maximum value of the wave height as baseline, finding out the pressure corresponding to 70% of the maximum wave height in accordance with the pressure rising direction of X-axle, which respresents the DBP;
(f) correcting the measured SBP and DBP according to the clinic correcting procedure to ensure accuracy.

Since the inventive apparatus system is equipped with a wristlet, air bag, barometer, central processing unit (CPU), pump, air escape valve, operating software needed in the steps stated above, SBP and DBP can be measured in advance according to the above-mentioned steps. After that, the inventive BP waveform piezoelectric sensor is used to obtain accurate BP waveforms, and then PMBP that is significant in medical science is estimated according to formula (1) to (3).

According to the present invention, haert rate variablilty and autonomic nervus system can be measured through using the non-invasive BP pluse waves and following the steps below:
(a) collecting continuously testees' BP waveform within the test period (i.e. 5 min. or 24 hrs);
(b) selecting a specific point (i.e. primary peak) on the BP waveform as the reference to the waveform;
(c) estimating the time interval (i.e. primary peak-to-primary peak interval) between reference point of each waveform and next one;
(d) estimating the mean of time interval and Standard Deviation within tesing period;
(e) using Fast Fourier Transform to transform time interval to a spectrum, and figure out High Frequency Component (HF, 0.15-0.4 Hz), Low Frequency Components (LF, 0.04-0.15 Hz), Very Low Frequency (VLF, 0.0-0.04 Hz), and Total Power;
(f) defining the mean of time interval within (d) as heartbeat period, its reciprocal as heart rate (IR); defining the standard deviation of time interval within (d) as heart rate variability (HRV); defining heart rate variability (HRV) within (f) and total power within (e) as index of automatic nervus total activity; calculating LF % (=LF/(LF+HF)*100%) and HF % (=HF/(LF+HF)*100%) according to the parameters within (e), defining LF % as the index of sympathetic nervus activity and HF % as index of parasympathetic nervus activity, and defining LF/HF as the index of sympathetic-parasympathetic nervus balance. For deatils on the above-mentioned HRV analysis approaches (including time domain and frequency domain), definition of their parameters, and the relationship with automatic nersus system, refer to “Task Force of the European Society of Cardiology and the North American Society of Pacing and Electrophysiology. Heart rate variability: standards of measurement, physiological interpretation, and clinical use. Circulation. 1996; 93: 1043-1065.”

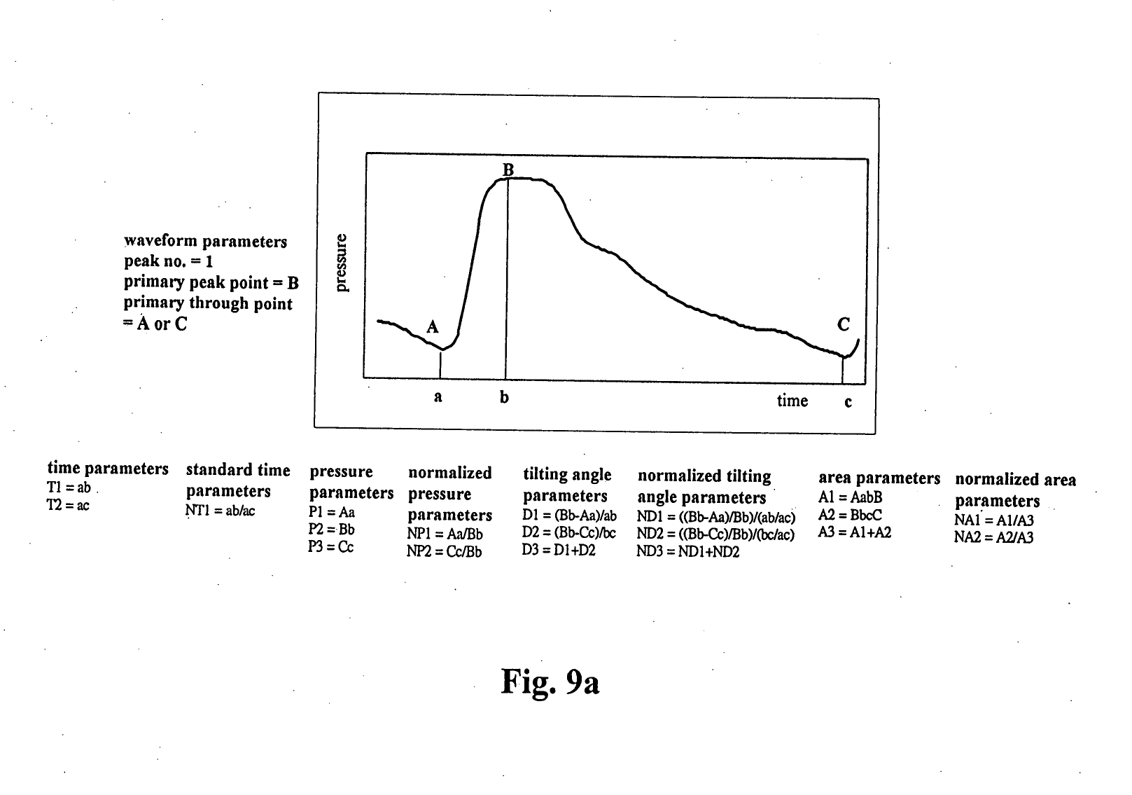

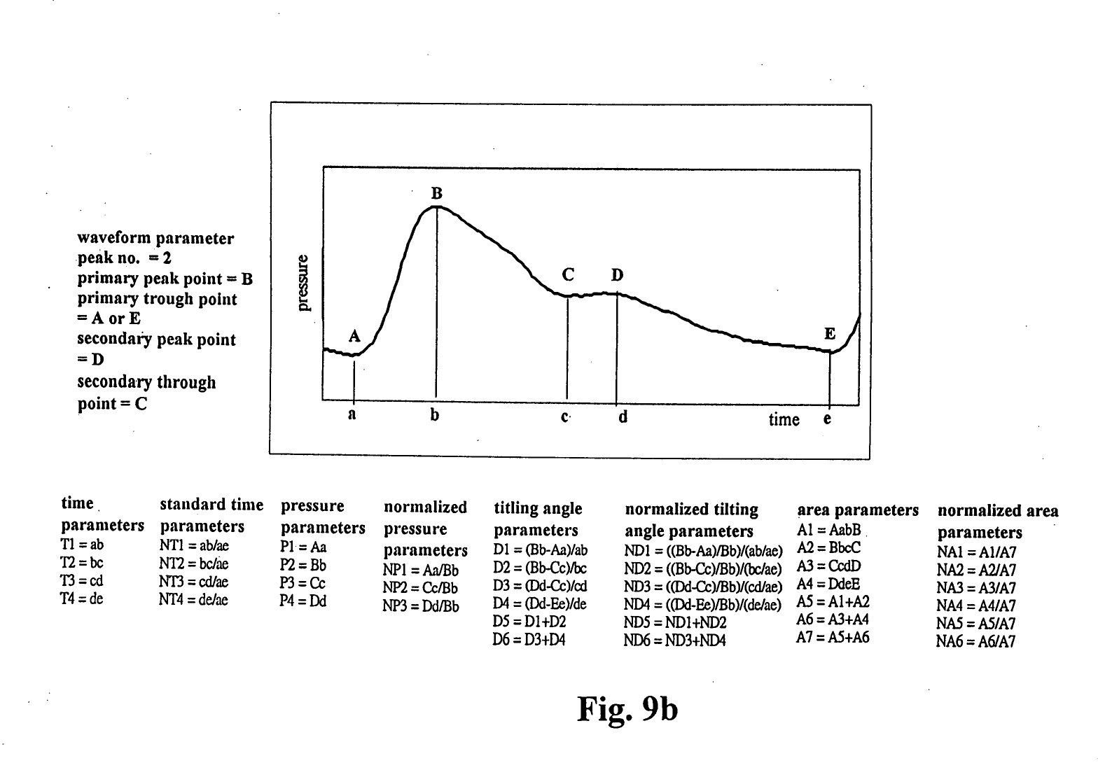

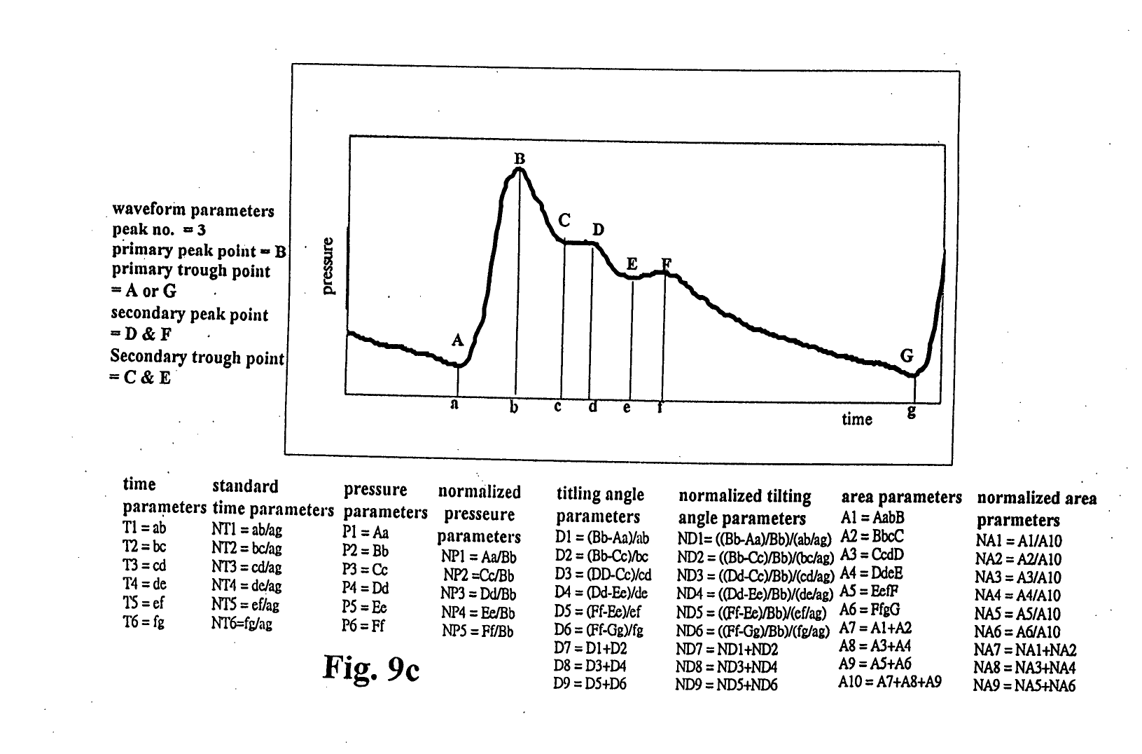

Mostly, the wrist radial artery BP waveform can be divided into one-waveform (in FIG. 9a), two-waveform (in FIG. 9b), and three-waveform (in FIG. 9c). According to the present invention, as shown in FIG. 9a to 9c and Chart 1, identifying testees can be achieved through using the above-mentioned non-invasive, accurate BP waveform measuring technique and following the steps below:
(a) collecting continuously testees' BP waveform (i.e. 50 sec. or 1 min.) regularly;
(b) finding out the starting point (trough) and endpoint (also the starting point of next waveform) of each BP waveform and each peak and trough point as shown in FIG. 9a to 9c;
(c) estimating the peak numbers, time, pressire, tilting angle, area, and their normalized characteristic parameters of each BP waveform (referring to Chart 1) as shown in FIG. 9a to 9c;
(d) estimating the mean of parameters within the test peiod, and then defining them as the characteristic baseline of testees' BP waveform;
(e) Next time, if the unknown testee's BP waveform characteristic parameters (d) has a certain degree of Similarity with characteristic baseline, the testee can be identified as the same one as described in
(a)˜(d) above, otherwise the testee is viewed as a different one;
(f) In a group (i.e. more than two) whose BP waveform characteristic baseline have been measured, if a person with unknown identification undergoes a test, the person's BP waveform characteristic parameters can be compared with individual data of the group one by one to choose one having high similarity (a certain degree of similarity), so that whether the testee is the identified one can be judged or not; the certain degree of similarity as addressed above can be regulated according to experiments and identification requirement.

An embodiment of the present invention is equally weighting all characteristic parameters of BP waveform, and defining the similarity of each parameter within positive/negative percentage (i.e. ±20%) of characteristic baseline. When a certain numbers of the parameter, which exceeds predetermined ratio (i.e. 80%), satisfies parameter similarity, this means that the criterion of total similarity can be attained, and that the two set of BP waveform data come from the same person. Said parameter similarity can be defined as ±10% of characteristic baseline or lower to increase soundness of personal identification, or be deifned as ±30% or more to decrease soundness of personal identification; similarly, the ratio of parameter numbers corresponding to simularity can be defined as 90% or more to increase soundness of personal identification, or be defined as 70% or lower to decrease soundness of personal identification.

Another embodiment of the present invention is defining the peak number (normally 1.0 to 3.0) and normalized time, pressure, tilting angle, and area characteristic parameters as shown in FIG. 9a to 9c, which are important parameters that satisfy similarity. Other non-normalized time, pressure, tilting angle, and area parameters are viewed as unimportant parameters. When the insignificant parameter that satiffies parameter similarity (i.e. between ±20% of characteristic baseline) exceeds the predetermined ratio (i.e. 80%), the two sets of BP waveform data can be indeitified as coming from the same person. When the insignificant parameter that satisfies parameter similarity is less than the predetermined ratio (i.e. 80%), the two sets of BP waveform data can be still indeitified as coming from the same person; otherwise, they are identified as coming from different persons. As described above, artery BP waveform may change slightly owing to emotional and physiological factors (i.e. HR accelates. and BP increase when feeling nervus, angry, or having a favor. However, the present invention have found that the Normalized time, pressure, tilting angle, and area characteristic parameters are constant and can serve as individualized characteristics. Comparing the non-normalized and normalized characteristic parameters simultaneously can enhance accuracy of personal identification.

The normalization procedure as described above conforms to conventional mathematic or statistic normalization procedure. Its objective is to transform dimension parameters into dimensionless parameters. As shown in FIG. 8a to 8c and Chart 1, a preferred normalization procedure of the present invention is that each time parameter devided by the period (that is, peak-peak interval) of BP waveform; each pressure parameter divided by the peak height of BP waveform (referring to the definitions in FIG. 3c); the denominator of each tilting angle parameter divided by the peak height of BP waveform (referring to the definitions in FIG. 3c), and its numerator divided by the-period (that is, peak-peak interval) of BP waveform; each area parameter divided by the total area of BP waveform. Due to the influence of natural aging and acute and chronic diseases, each person's BP waveform may gradually change with time. Accordingly, the BP waveform characteristic baseline described above should be measured and updated periodically (i.e. in one or two years) to ensure accuracy of personal identification.

CHART 1
Definition of BP Waveform

| Parameter/ | | | |
|---|---|---|---|
| Parameter | Caracteristic Parameter | | |
| Definition | 1-Waveform (FIG. 8a) | 2-Waveform (FIG. 8b) | 3-Waveform (FIG. 8c) |
| Peak num. | 1 | 2 | 3 |
| Primary Peak | B | B | B |
| Point | | | |
| Primary Trough | A, C | A, E | A, G |
| Point | | | |
| Secondary Peak | No | D | D, F |
| Point | | | |
| Secondary Trough | No | C | C, E |
| Point | | | |
| Time Parameter | T1 = ab | T1 = ab | T1 = ab |
| | T2 = ac | T2 = bc | T2 = bc |
| | | T3 = cd | T3 = cd |
| | | T4 = de | T4 = de |
| | | | T5 = ef |
| | | | T6 = fg |
| Normalized Time | NT1 = ab/ac | NT1 = ab/ae | NT1 = ab/ag |
| Parameter | | NT2 = bc/ae | NT2 = bc/ag |
| | | NT3 = cd/ae | NT3 = cd/ag |
| | | NT4 = de/ae | NT4 = de/ag |
| | | | NT5 = ef/ag |
| | | | NT6 = fg/ag |
| Pressure | P1 = Aa | P1 = Aa | P1 = Aa |
| Parameter | P2 = Bb | P2 = Bb | P2 = Bb |
| | P3 = Cc | P3 = Cc | P3 = Cc |
| | | P4 = Dd | P4 = Dd |
| | | | P5 = Ee |
| | | | P6 = Ff |
| Normalized | NP1 = Aa/Bb | NP1 = Aa/Bb | NP1 = Aa/Bb |
| Pressure | NP2 = Cc/Bb | NP2 = Cc/Bb | NP2 = Cc/Bb |
| Parameter | | NP3 = Dd/Bb | NP3 = Dd/Bb |
| | | | NP4 = Ee/Bb |
| | | | NP5 = Ff/Bb |
| Tilting Angle | D1 = (Bb − Aa)/ab | D1 = (Bb − Aa)/ab | D1 = (Bb − Aa)/ab |
| Parameter | D2 = (Bb − Cc)/bc | D2 = (Bb − Cc)/bc | D2 = (Bb − Cc)/bc |
| | D3 = D1 + D2 | D3 = (Dd − Cc)/cd | D3 = (Cc − Dd)/cd |
| | | D4 = (Dd − Ee)/de | D4 = (Dd − Ee)/de |
| | | D5 = D1 + D2 | D5 = (Ff − Ee)/ef |
| | | D6 = D3 + D4 | D6 = (Ff − Aa)/fg |
| | | | D7 = D1 + D2 |
| | | | D8 = D3 + D4 |
| | | | D9 = D5 + D6 |
| Normalized | ND1 = ((Bb − Aa)/ | ND1 = ((Bb − Aa)/ | ND1 = ((Bb − Aa)/ |
| Tilting Angle | Bb)/(ab/ac) | Bb)/(ab/ae) | Bb)/(ab/ag) |
| Parameter | ND2 = ((Bb − Cc)/ | ND2 = ((Bb − Cc)/ | ND2 = ((Bb − Cc)/ |
| | Bb)/(bc/ac) | Bb)/(bc/ae) | Bb)/(bc/ag) |
| | ND3 = ND1 + ND2 | ND3 = ((Dd − Cc)/ | ND3 = ((Cc − Dd)/ |
| | | Bb)/(cd/ae) | Bb)/(cd/ag) |
| | | ND4 = ((Dd − Ee)/ | ND4 = ((Dd − Ee)/ |
| | | Bb)/(de/ae) | Bb)/(de/ag) |
| | | ND5 = ND1 + ND2 | ND5 = ((Ff − Ee)/ |
| | | ND6 = ND3 + ND4 | Bb)/(ef/ag) |
| | | | ND6 = ((Ff − Aa)/ |
| | | | Bb)/(fg/ag) |
| | | | ND7 = ND1 + ND2 |
| | | | ND8 = ND3 + ND4 |
| Area Parameter | A1 = AabB | A1 = AabB | A1 = AabB |
| | A2 = BbcC | A2 = BbcC | A2 = BbcC |
| | A3 = A1 + A2 | A3 = CcdD | A3 = CcdD |
| | | A4 = DdeF | A4 = DdeE |
| | | A5 = A1 + A2 | A5 = EefF |
| | | A6 = A3 + A4 | A6 = FfgG |
| | | A7 = A5 + A6 | A7 = A1 + A2 |
| | | | A8 = A3 + A4 |
| | | | A9 = A5 + A6 |
| | | | A10 = A7 + A8 + A9 |
| Normalized Area | NA1 = A1/A3 | NA1 = A1/A7 | NA1 = A1/A10 |
| Parameter | NA2 = A2/A3 | NA2 = A2/A7 | NA2 = A2/A10 |
| | | NA3 = A3/A7 | NA3 = A3/A10 |
| | | NA4 = A4/A7 | NA4 = A4/A10 |
| | | NA5 = A5/A7 | NA5 = A5/A10 |
| | | NA6 = A6/A7 | NA6 = A6/A10 |
| | | | NA7 = NA1 + NA2 |
| | | | NA8 = NA3 + NA4 |
| | | | ND9 = ND5 + ND6 |

Note:
(1) Lower-case a, b, c, d, e, f, g represent time; unit of measurement is second.
(2) Upper-case A, B, C, D, E, F, G represent the spots on the BP waveform curve; unit of the coordinate axis is (pressure, sec) or (mV, sec.)
(3) Area Parameter (i.e. AabB) is an integral area, which is formed by curves and four angles of points (also can be simply defined as a trapezoid area with four angles of points)

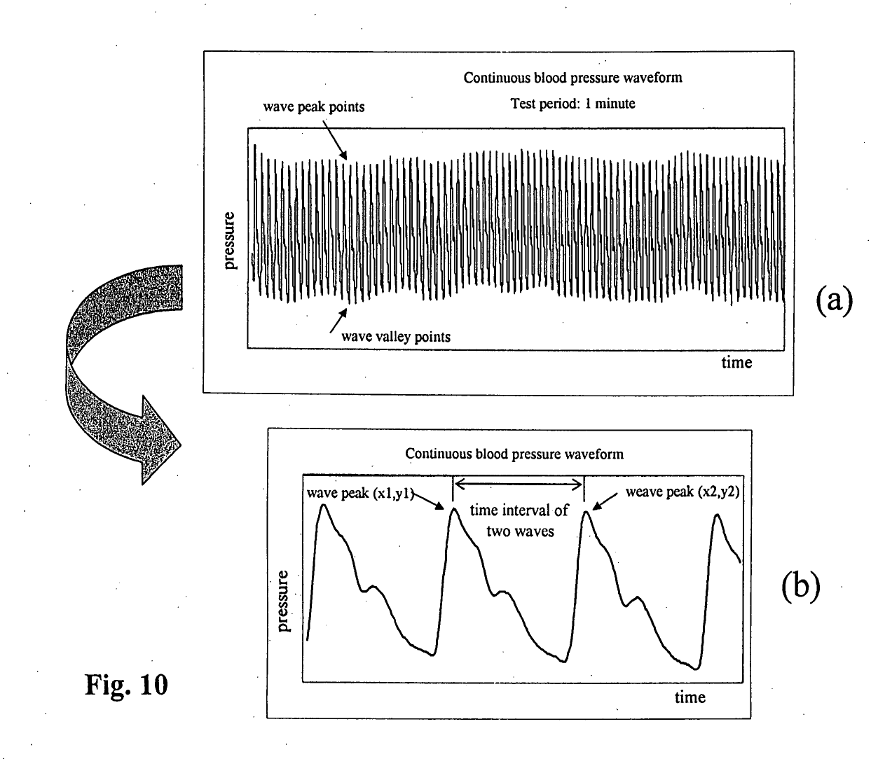

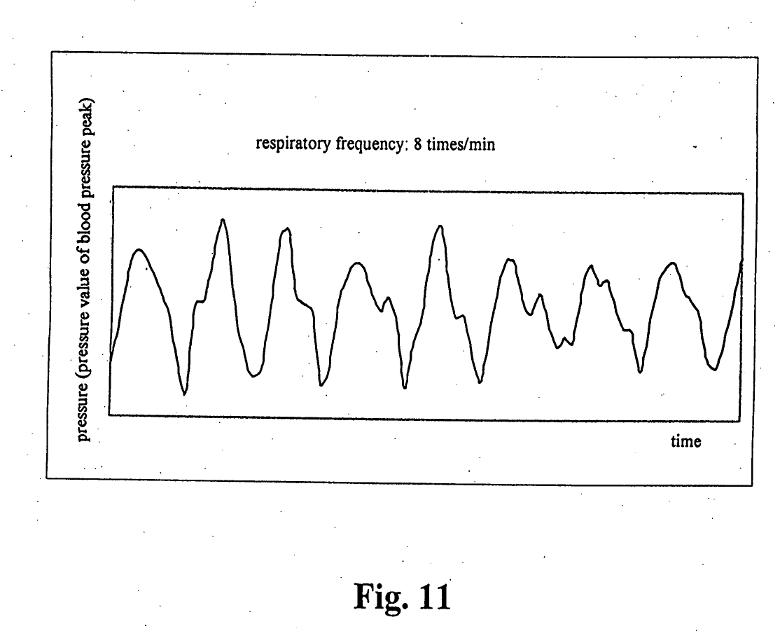

According to the present invention, as shown in FIGS. 10, 11, and 12, the above-mentioned non-invasive accurate BP waveform measuring technique can be performed by following the procedures below to measure respiratory waveform and frequency:
(a) collecting continuously testees' BP waveform within the test period (i.e. 1˜10 mins), and drawing a X-Y diagram (FIG 10a) accordingly, wherein X-value represents time, Y-value represents voltage or pressure;
(b) finding out the X value (time value) and Y value (voltage or pressure value) of primary peak Point of each waveform (referring to FIG. 10b),
(c) using the primary peak point data in (b) to draw a continuous XY diagram (FIG. 11), which represents testees' respiratory waveforms;
(d) estimating the number (i.e. eight) of waveforms on a regular time (i.e. one min.) in (c), the estimated number represents the respirotary frequency or respiratory rate (i.e. eight times per minute).

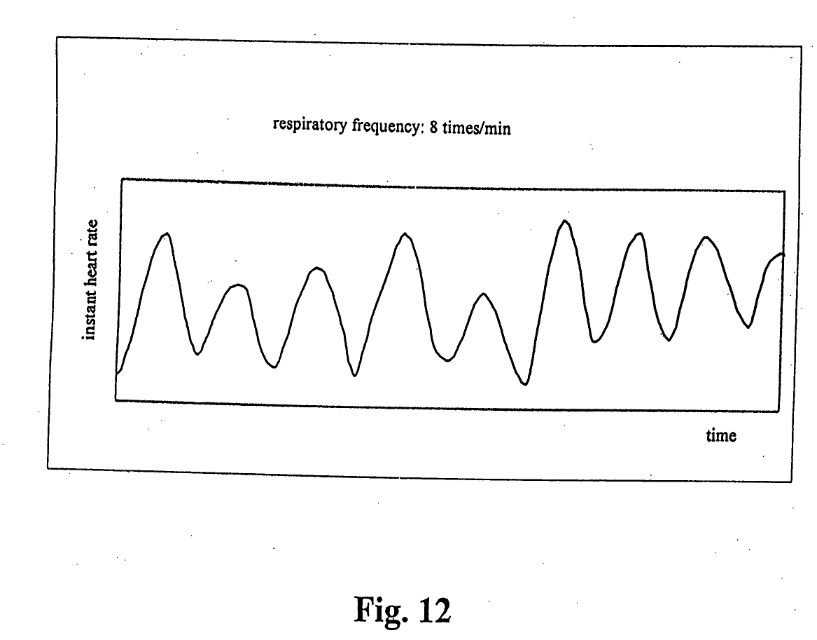

In the procedure (b) described above, primary peak serves as basis of all calculation; if primary trough (the starting point of each waveform, also the lowest point) or other specific point of BP waveform is used as basis of the calculation, respiratory waveform and frequency (or respiratory rate) can still be obtained through the procedure described above. In addition, referring to FIGS. 10b and 12, if the testee's primary peak-primary peak time interval (referring to FIG. 10b;) is calculated, its reciprocal (also instant heart rate) is referred as Y value, and the corresponding time is referred as X value, then a continuous X-Y diagram (FIG. 12) is drawn, which represents the testss's respiratory waveforms. Similarly, the waveform number (i.e. eight) in a certain period in the respiratory waveform diagram is calculated, representing the respirotary frequency or respiratory rate (i.e. eight times per minute).

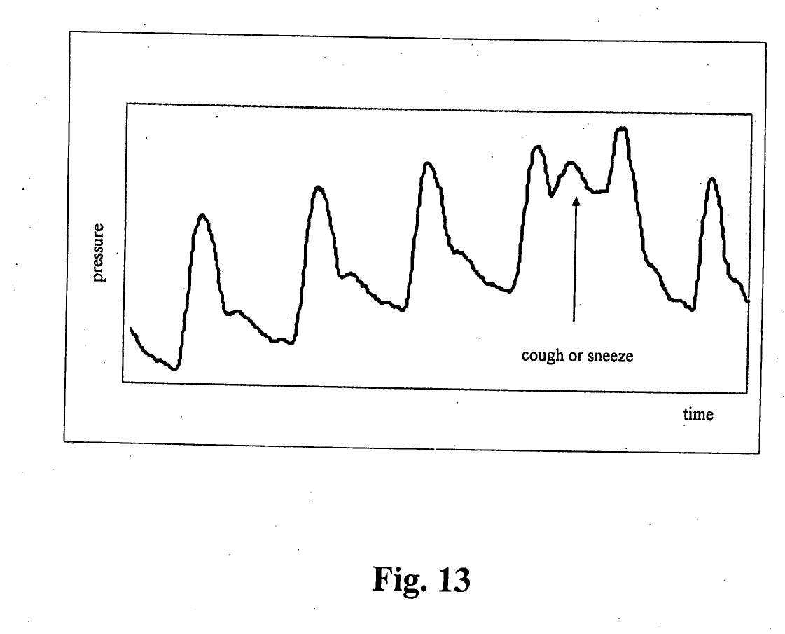

According to FIG. 13, symptoms of cough or sneeze can be monitored through using the above-mentioned non-invasive, accurate BP waveform measuring technique and following the procedures below:
(a) collecting continuously testees' BP waveform;
(b) estimating the mean and standard division of three parameters (primary peak-primary peak time interval, pressure value of primary peak, and pressure value of primary trough) of BP waveform within initial stage (i.e. one min.), and defining the mean as baseline of the three parameters;
(c) monitoring the three parameters of BP waveform all the time within the test period; if one (or more) of them deviate itself from the baseline above the perdetermined multiple (i.e. triple) of standard deviation, the waveform can be called as suspicious irregular data point;
(d) if the suspicious irregular data point occurs with a certain frequency (i.e. occurring once every four waveform period), it can then be ignored;
(e) if the suspicious irregular data point occurs randomly (without regular frequency), the data point is defined as cough or sneeze point;
(f) regularly compiling statistics of cough or sneeze points in (e), so that the cough or sneeze frequency (i.e. three times per minute) can be obtained.

The predetermined Standard Deviation multiple as described above, can be tested by body experiments. For example, as to slighter cough to which the degree of its BP waveform deviating from the mean is lower, therefore, the predetermined multiple of standard deviation can be set within 3 to 4; on the contrary, heavier cough or sneeze can be set within 4 to 6. Moreover, the suspicious irregular data point with regular frequency described above is often caused by cardiac arrhythmias, and thus should be excluded.

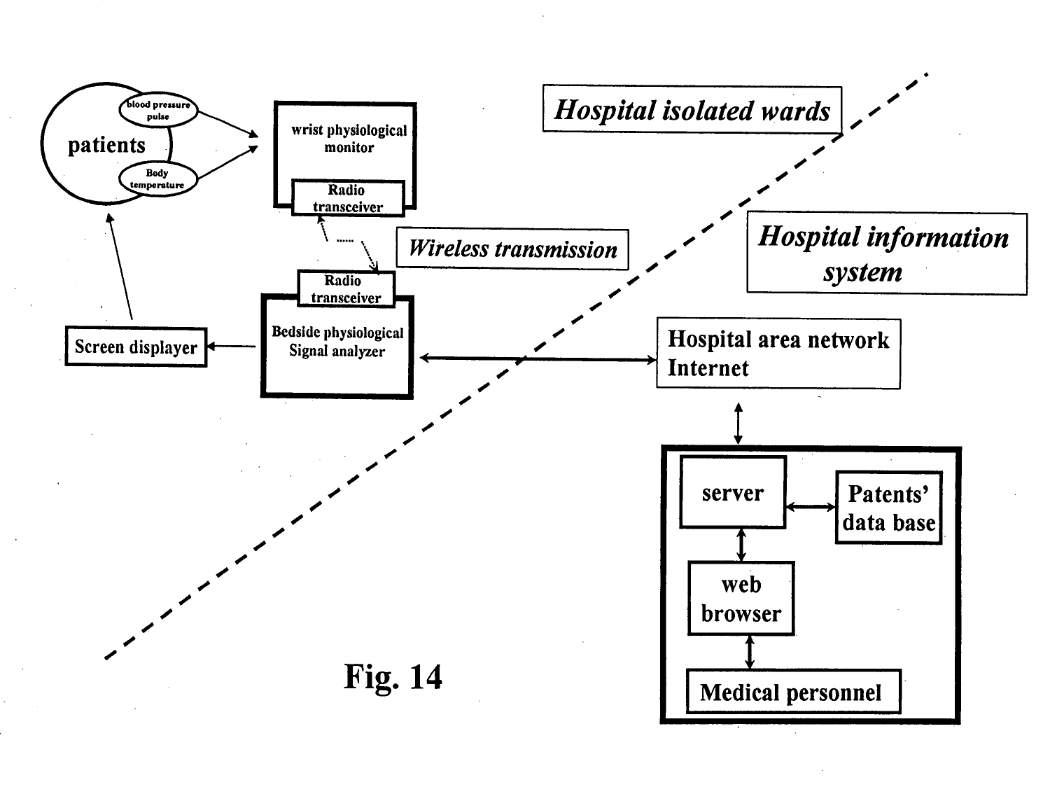

The present invention further provides a new physiological signal monitoring apparatus system for hospital quarantine monitoring (as shown in FIG. 14), including:
(a) The non-invasive, accurate BP waveform measuring technique and its apparatus system thereof addressed above;
(b) conventional body temperature measuring technique, by which a temperature sensor is disposed onto the wristlet of the apparatus (a);
(c) conventional wireless signals transmission technique, by which a set of wireless transmission module is installed in the host (a), and another set of wireless module is installed in the bedside analyzer capable of receiving patients' physiological signals;
(d) using the apparatus system (a) addressed above to measure the HR, BP (SBP, DBP, PMBP), repiratory waveform and frequency of an isolated patient, and judge if the patient has palpitations, rapid BP variations, tachypnoea, cough or sneeze accordingly;
(e) using the temperature sensor in (b) to measure patients' wrist temperature, and judge if the patient has favor accordingly;
(f) using the wireless transmission module (c) to transmit physiological signals in (d) and (e) from the host (a) to bedside analyzer (c);
(g) using the bedside analyzer to send the patient's physiological signals and analysis results out of the ward through Local Area Network (LAN) or Internet.

The above-mentioned conventional body temperature measuring technique may employ a small thermocouple, a Resistor-type electric device, or an infrared photo-electric device. The three devices are all thermal-sensitive devices, which can transform body temperature into voltage or current signals. To enhance accuracy of measuring body temperature and reduce the time for achieving temperature balance. In addition to said thermal-sensitive devices, a piece of metal can be added onto the wristlet with its metal covering uncovered outside the wristlet cloth. It can have direct contact with wrist skin when being put on one's wrist for heat conduction. This metal film is connected to thermal-sensitive devices in the wristlet, while the thermal-sensitive devices are connected to the circuit module of the present invention, which forms a complete body temperature measuring system. The above-mentioned wireless transmission technique may employ a set of commercial radio frequency (RF) wireless modules with its frequency domain within frequently used ISM band (that is, [Industry, Science, Medicine] sharing band), wherein the Internet Protocol used most is Blue Tooth (2.4 GHz), Wi-Fi (including IEEE 802.11b, 802.11a, and 802.11g, 2.4˜5.6 GHz), and Low Frequency ISM(433˜915 MHz). In addition, US Food and Drug Administration (FDA) also sets a WMTS channel (608˜1429 MHz) for the use of medical appliances. RF wireless modules are usually operated by the way of two-way transmission.

In view of the example described above, one of the wireless transmission modules are installed in the wrist host of the present invention, and another is installed in the bedside analyzer in isolation wards. Said bedside analyzer can be a desk-top computer or laptop, or other devices with the computing, storage, display, and transmission function. The wireless transmission module can be connected to the bedside analyzer through standard interface (i.e. RS-232 (COM Port), USB, IEEE 1394). If a patient's HR exceeds normal HR (i.e. 100 times per minute at most) as defined under general medical definition, this persopn is judged as having palpitations; the judgement of cough or sneeze have been desribed above and will be omitted here. In addition, the maximum of the respirotary frequency can be defined (i.e. 20 times per minute). When a testee's respirotary frequency exceeds this, he can be judged as having tachypnoea;

In addition, according to the general medical norm, when one's body temperature exceeds 38° C., this person can be identified as having favor. After the patient's physiological signals are transmistted to a bedside analyzer, they are stored, analyzed, and displayed, and then delivered out of a ward (i.e. transmitted to nursing stations or patient's information server) through Local Area Network (LAN) or Internet in the hospital.

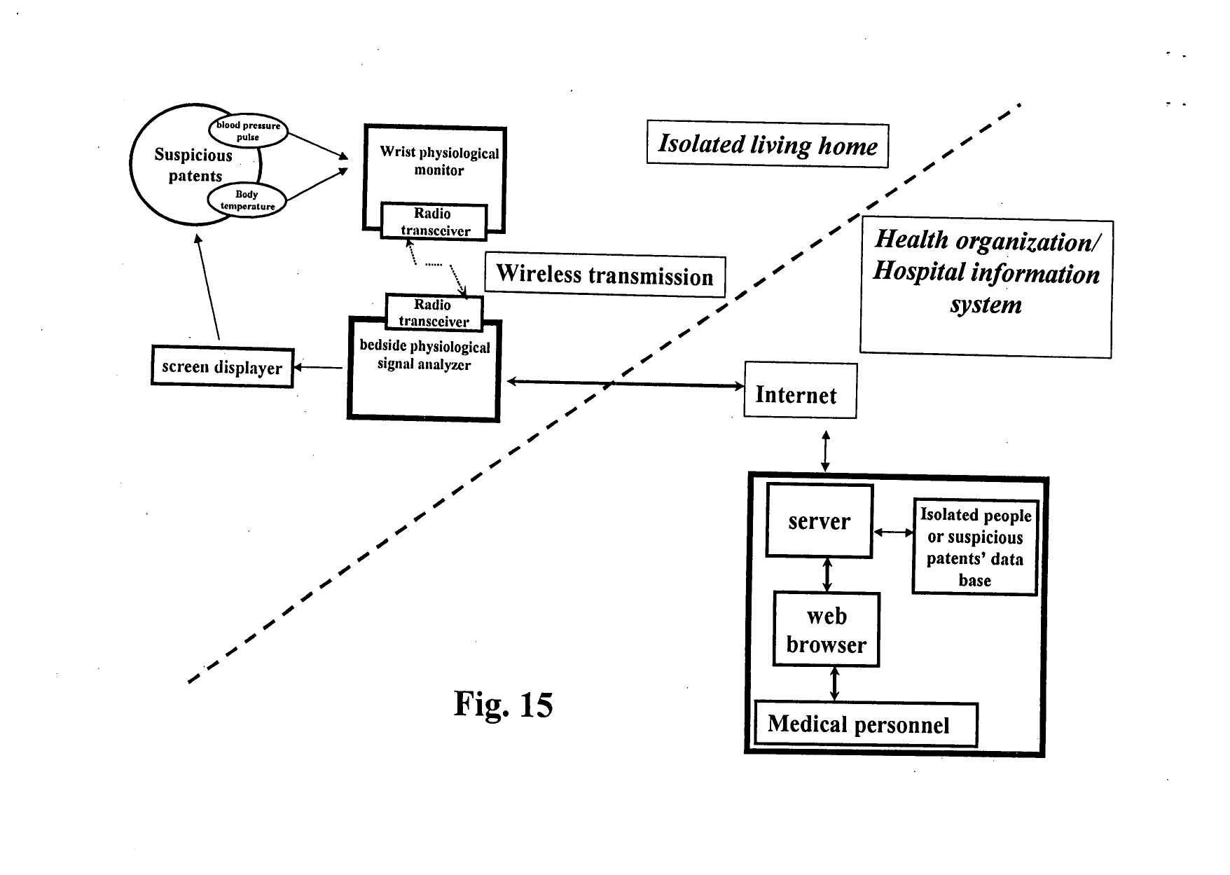

The present invention further provides an apparatus system for uses in home quarantine monitoring (as shown in FIG. 15), including:
(a) The non-invasive, accurate BP waveform measuring technique and its apparatus system thereof addressed above;
(b) using the apparatus system (a) to measure home quarantine patients' physiological signals including HR, BP (SBP, DBP, and PMBP), BP waveform, respiratory waveform and frequency, and judge if the persone has symptoms such as palpitations, rapid BP variations, tachypnoea, cough or sneeze;
(c) using conventional body temperature measuring techniques to measure the person's body temperature, and then judge if the person has symptoms such as favor accordingly;
(d) using conventional wireless transssion techniques to regularly transmit physiological signals (a) to (c) to the signal processor at home;
(e) after said signal processor stores and analyzes (or displays) physiological information, the processed data are delivered to health organizations (i.e. Department of Health, Health Administration Organization, or hospitals) through the Internet or Modem;
(f) relevant health organizations regularly compile statistic on demographic data of the healthy, sick, and recovered.

To prevent isolated patients from leaving home without permission, relevant health organizations may give commands to a bedside analyzer at any time through the Internet or Modem. Then, the bedside analyzer delivers the command to a wrist physiological monitor through wireless transmission. If an isolated patient stays at home at that moment, after hearing an alarm sound from a bedside analyzer or a wrist physiological monitor (through the included buzzer, the patient should put on the wrist physiological monitor to proceed tests. If the isolated patient leaves home without permission, relevant health organization should determine if the patient leaves without permission and proceed subsequent control measures if they did not receive any physiological signals after a period (i.e. within 10 mins.) of giving test commands. If the isolated patient leaves home without permission, while somebody else does the test instead, the health organization can determine whether the data comes from the same persone through the personal identification technique proposed in the present invention, and adopts necessary control measures further. Said signal processor can be a desk-top computer, laptop, Personal Digital Assistant (PDA), or other devices with the transmission, storage, analysis, and display function. The definition of the so-called healthy, sick, and recovered patients can be adjusted by the Health Authority according to the regulations on physiological parameters such as BP change, respirotary frequency, cough and sneeze, and body temperature. Also, descriptions regarding body temperature, BP, wireless transmission and Internet transmission techniques have been detailed above, and will be omitted.

The following examples of the present invention have been described for illustrative purposes, but they are not limited in the present invention.

### EXAMPLE 1

### A Non-Invasive Wrist Radial Artery BP Waveform Measuring Apparatus System

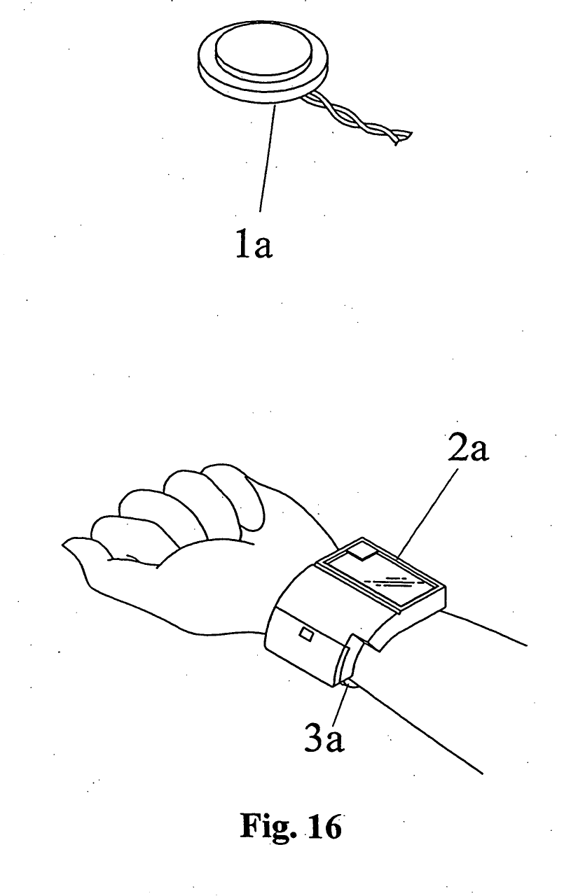

The apparatus system in the present example is composed of components listed below (also shown in FIG. 16, each component corresponds to a reference number):
(a) a piezoelectric sensor 1a comprising a strain gauge, which is a circular-shaped thin film (its length is 5 mm and thickness is 3 mm), including circuits for signal filter, maginification, adjustment, and temperature compensation. In addition to the circular0shaped sensor, a wire is connected to the circuit module of wrist physiological monitor (will be described later). The function of said wire is to provide power source for the piezoelectric sensor and transmit piezoelectric signals to the circuit module.
(b) a wrist physiological monitor 2a, which includes an air pump, an air escape valve, standard 2A dry cells (two), RS232 port, a barometer, and a circuit module comprising a central processing unit (CPU), memory, and signal processor (filter, magnification, and adjustment); said host includes an casing with an upper and lower cover; said casing has a cramp and an eyelet, so that it can be fixed onto the wristlet(will be described later); said upper cover includes buttons for operating the present apparatus;
(c) a wristlet 3a, which includes an air bag and a U-shaped plastic thin film; said plastic thin film has a cramp and an eyelet, so that it can be connected with the lower cover of said wrist host; the length of said wristlet is longer than the length of one's wrist, so that the remaining, after using said wristlet to encircle wrist in a week, can be folded back and fastened with the velcro.

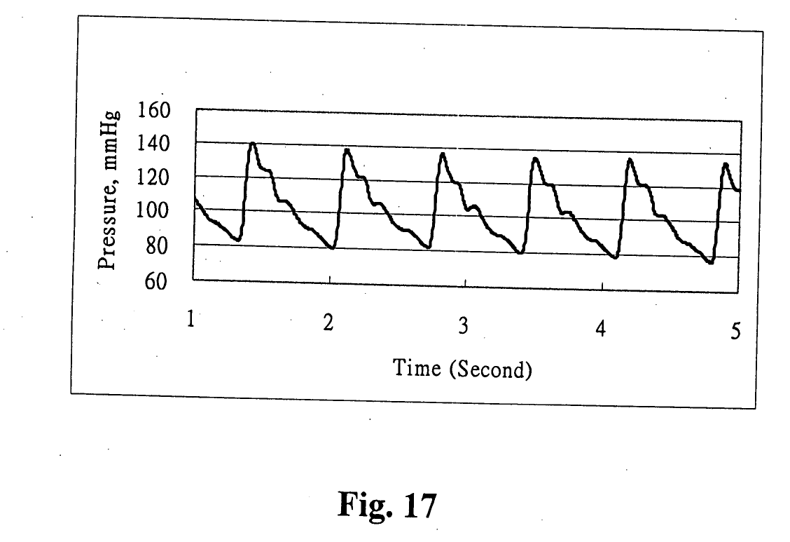

The operating procedures of the present apparatus system are described as follows:
(d) using fingers to feel the wrist pulse (no matter left hand or right hand) of a healthy voluntary testee, and measure correct radial artery position;
(e) fastening circular-shaped piezoelectric sensor in (a) upon the testee's radial artery with adhesive tapes;
(f) putting the wrist host in (b) with the wristlet in (c) upon the testee's wrist;
(g) pressing the “On” button on the apparatus (b), then starting a test; at this moment, the operating software gives commands to the air pump to start to pump airs (the air escape valve closes at this moment; when the air bag is pressurized to 200 mmHg (barometer value), the operating software gives commands to the air escape valve to slow down air escape. Meanwhile, said operating software and circuit module reads and stores the barometer value and pulse wave signals of the piezoelectric sensor in a speed of 500 data point per second, while said operating software estimates the peak height (peak-peak interval) of each pulse wave during the period of air escape (200 descreased to 30 mmHg, about 20 secs.), and determines the air pressure value of the maximum peak height; such value (93 mmHg) represents optimal testing air pressure (OTAP);
(h) said: operating software gives commands to the air pump, and air pressure of said air bag is increased from 30 mmHg to optimal testing air pressure (93 mmHg), after that, similarly, operating software and circuit module reads the pulse wave signals of said piezoelectric sensor in a speed of 500 data point per second; the sample is taken in five seconds, while the data and its corresponding time value (obtained from Real-Time Clock) are stored in the memory of said circuit module;
(i) starting up a personal computer (PC), while the RS-232 (COM Port) connector is connected to the RS-232 post of the wrist host. The PC operating system gives commands to the circuit module of wrist host and its operating software, and starts to process data download;
(j) said operating software drew a X-Y diagram according to the downloaded data, wherein X-axle represents time (unit: sec.), Y-axle represents the BP value of piezoelectric sensor (unit: mmHg); the results are shown in FIG. 17.

### EXAMPLE 2

### A Non-Invasive Wrist Radial Artery BP Waveform Measuring Apparatus System for Uses in PMBP Measurment

The hardware, software, operating procedures, and voluntary testees in the present example are similar to the ones in Example 1, but the piezoelectric device is changed from the 5-mm circular-shaped sensor described in Example 1 to a 5-mm ceramic PZT sensor (in the direction of hand length)×20-mm (W) (in the direction of hand width)×2-mm (D); moreover, a LCD display is added into the wrist host. After following the procesure (g) of Example 1 to obtain the maximum peak height (pressure value is 93 mmHg at that time) during air escape, implement the steps described below:
(a) the operating software of a wrist host uses the maximum wave as baseline to find out the BP waveform corresponding to 50% of the maximum wave in the direction against pressure escape (or toward the air pressure rising direction), the pressure value (138 mmHg) represents SBP; the operating software of a wrist host uses the maximum wave as baseline to find out BP waveform corresponding to 70% of the maximum wave in the direction of pressure escape (toward the air pressure falling direction), the pressure value (83 mmHg) represents DBP;
(b) moreover, according to body experiments, the SBP (a) needs to be corrected decreasingly about 25 mmHg, and DBP needs to be corrected decreasingly about 10 mmHg, so that accurate SBP (113 mmHg) and DBP (73 mmHg) can be obtained;
(c) estimating the mean (86 mmHg) of PMBP within a 5 sec. test according to Formula (1)˜(3) in the detailed description of the present invention;
(d) the LCD display of a wrist host shows the mean (86 mmHg) of PMBP;
(e) moreover, according to related body experiments, it can be found that the measured SBP, DBP, and PMBP of the healthy, voluntary testees, through measurement of invasive wrist artery intubating, are 110, 68, and 85 mmHg, respectively. The results show that the SBP, DBP, and PMBP measured by the standard invasive wrist artery intubating measuring method has high similarity as compared with ones measured by the present non-invasive radial artery BP waveform measuring method.

### EXAMPLE 3

### A Non-Invasive Wrist Radial Artery BP Waveform Measuring Apparatus System for Uses in Heart Rate Variability and Automonic Nervus System Measurements

The apparatus hardware in the present example is similar to those in Example 2, except for the participation of two healthy voluntary testees. Moreover, the piezoelectric sensor is changed from PZT piezoelectric materials as described in Example 2 to PVDF materials; its size is 5-mm (L) (parallel to hand length)×20-mm (W) (parallel to hand width)×0.5 mm (D). In addition, this piezoelectric sensor is installed inside the wristlet in advance, between top of air bag and outer cloth of wristlet; wherein the tail connector (which is composed of soft printed circuit board) is connected to the circuit module in the host through the interior part of said wristlet, a small opening, and the lower casing of wrist host. The signal processing circuit and LCD display of the circuit module are the same as the ones in Example 2. To put on the apparatus, you only need to fasten the wrist host with the wristlet upon your wrist. After obtaining continuously BP waveforms according to the procedures in Example 1, implement the steps below:
(a) collecting continuously testees' BP waveforms within 5 mins.; the operating software of wrist host measures peak-peak time interval of each BP waveform within the test period;
(b) estimating testee's HRV parameter within Time Domain (that is, heart period, HR, and HRV according to the method addressed in “Task Force of the European Society of Cardiology and the North American Society of Pacing and Electrophysiology. Heart rate variability: standards of measurement, physiological interpretation, and clinical use. Circulation. 1996; 93:1043-1065”;
(c) estimating testee's HRV parameter within Frequency Domain (that is, Total Power, High Frequency (HF) component, Low Frequency (LF) component, and HF/LF ratio) according to the reference illustrated in (c);
(d) defining HRV within time domain and Toal Power within frequency domain as the index of automatic nervus activity according to the reference illustrated in (c); defining High Frequency (HF) component within frequency domain and HF percentage (HF/(CHF+LF))×100%) as the index of parasympathetic nervus activity; defining Low Freqency (LF) component within frequency domain and LF percentage (LF/(HF+LF))×100%) as the index of sympathetic nervus activity; defining LF/HF ratio as the index of sympathetic/parasympathetic nervus balance.
(e) Moreover, while the testee undergoes a BP waveform test as described above, he also undergoes a standard electrocardiograph (ECG) test in 5 mins. (connection method is standard three electrode process, Lead I, II, and III, and Lead II to be computed source), and each HRV parameter within time and frequency domain have been measured according to the reference as illustrated above.

(f) Test results are shown in Chart 2. The results of two healthy voluntary testees (code A, B) reveal that standard electrocardiograph (ECG) test method has a high similarity with compared to the HRV and automatic nervus activity index measured by the non-invasive radial artery BP waveform technique in the present invention.

CHART 2
Test Results of Heart Rate Variability and Autonomic Nervus
Activity Index

| | Testee code | | | |
|---|---|---|---|---|
| | A | | B | |
| | Test method | | | |
| | | Present | | Present |
| | ECG | invention | ECG | invention |
| | Test gesture | | | |
| | Lying face up | | Lying face up | |
| Heart Period (s) | 0.910 | 0.907 | 0.723 | 0.723 |
| HR (time/min) | 66 | 66 | 83 | 83 |
| HRV (ms); or Standard | 45.1 | 46.0 | 57.1 | 55.4 |
| Deviation | | | | |
| Total Power (ms * ms) | 2037 | 2120 | 3265 | 3073 |
| LF (ms * ms) | 420 | 426 | 413 | 418 |
| LF % | 63 | 61 | 32 | 31 |
| HF (ms * ms) | 252 | 275 | 894 | 943 |
| HF % | 38 | 39 | 68 | 69 |
| LF/HF | 1.67 | 1.55 | 0.46 | 0.44 |

### EXAMPLE 4

### For Uses in Personal Identification

The hardware of the present examples is similar to the ones of Example 2, but the composition of piezoelectric sensor is changed as follows:
(a) A soft printed circuit board, used as the base and basic circuit of the piezoelectric sensor; its main body size is 6 mm (L) (parallel to hand length)×20 mm (W) (parallel to hand width)×0.5 mm (D); its tail connector size is 6 mm×50 mm×0.5 mm;
(b) Three ceramic PZT piezoelectric devices, wherein each device size is 4 mm (L)×4 mm (W)×1 mm (D), which are aligned on the soft printed circuit board parallel to hand width with a 1-mm interval between a device and another device (referring to FIG. 5).

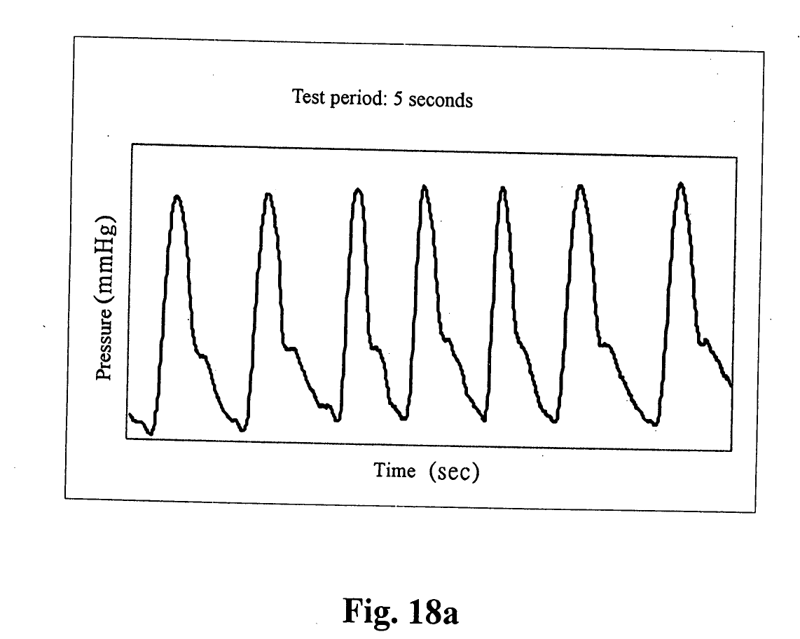

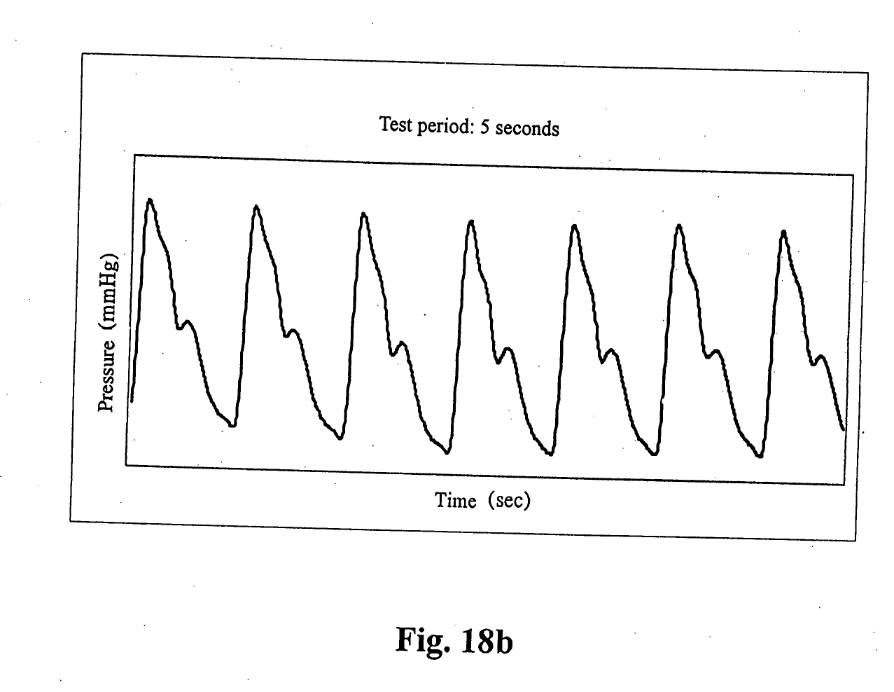

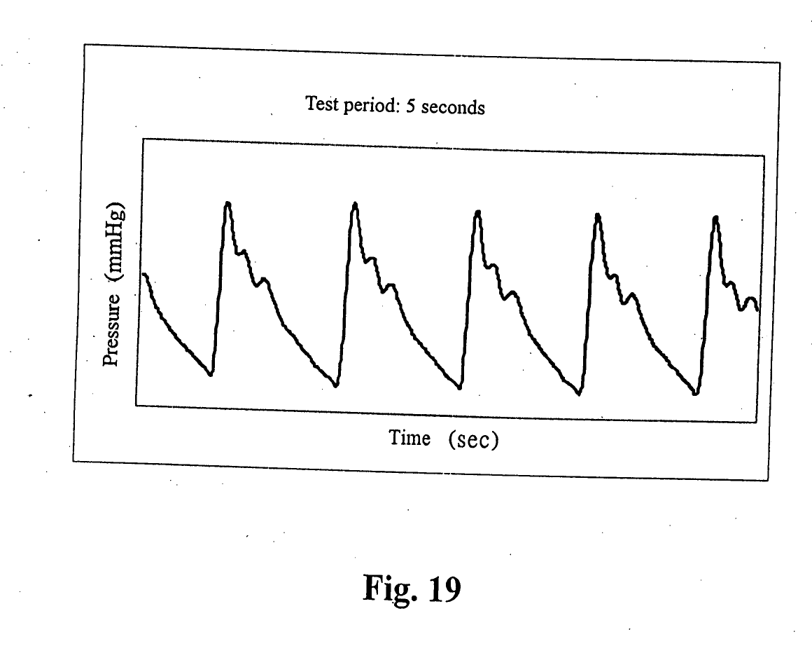

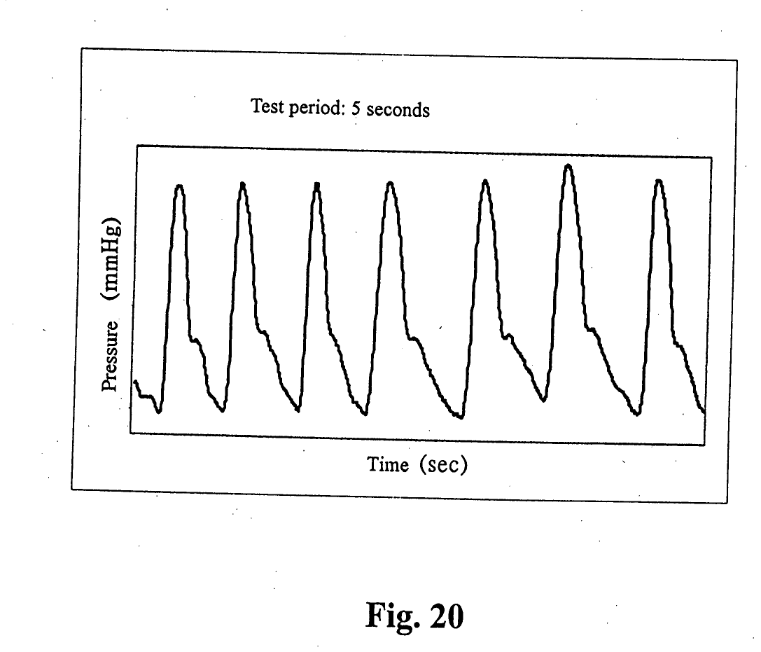

Said piezoelectric sensor is installed in the wristlet beforehand, the procedures are similar to the ones in Example 3; in addition, in addition to signal processing (filter, magnification, and adjustment) circuits, a multi-plexer is added onto the circuit module in front o the signal processing circuit. When a test is carried out, the operating software of wrist host drives the multi-plexer to obtain electric wave signals of each device on the piezoelectric sensor in sequence, and then measures the Peak Height (peak-trough) of BP waveform of the three devices. The operating software further chooses the piezoelectric device having the maximum peak height as the source of BP waveform data, while the other two devices are no longer used. After that, the operating software searches for optimal testing air pressure, as illustrated in Example 1, under the optimal testing air pressure, a test to continuously measure the BP waveform within 10 sec. is conducted. Testee A, B, and C has undergone a test (as shown in FIG. 17, 18, 19), the next day, one of the three testees with unknown identification has undergone a second test (FIG. 20); the test results including BP waveform and their parameters are summarized in FIG. 17 to 20 and Chart 3. From FIG. 18, 20 and Chart 3, it can be found that the three testees' BP waveform characteristic parameters are all different. Further analysis shows that the unknown testee has two-peak (two-peak) BP waveform, which seems different from testee C who has three-peak (three-peak) BP waveform (referring to FIG. 19, 20 and Chart 3). If the 41 characteristic parameters (including peak number, time, pressure, tilting angle, area, and their normalized characteristic parameters) of the 2-peak BP waveform are equally weighted, and the similarity of each parameter is defined within ±20% of the characteristic baseline, the unkown testee's 38 parameters (93%) satisfies with testee A's, only 25 parameters (61%) satisfies with testee B's; therefore, according to further judgement, the known testee should be testee A (the result is identical to the experiemt design).

CHART 3
Testee's BP waveorm
characteristic parameter

| | Testee/Parameter | | | |
|---|---|---|---|---|
| | A | B | C | Unknown |
| Peak num. | 2 | 2 | 3 | 2 |
| Primary Peak | B | B | B | B |
| Point | | | | |
| Primary | A, E | A, E | A, G | A, E |
| Trough Point | | | | |
| Secondary | D | D | D, F | D |
| Peak Point | | | | |
| Secondary | C | C | C, E | C |
| Trough Point | | | | |
| Time | T1 = 0.155 | T1 = 0.095 | T1 = 0.090 | T1 = 0.160 |
| Parameter | T2 = 0.195 | T2 = 0.230 | T2 = 0.100 | T2 = 0.165 |
| (sec) | T3 = 0.055 | T3 = 0.060 | T3 = 0.045 | T3 = 0.055 |
| | T4 = 0.345 | T4 = 0.360 | T4 = 0.100 | T4 = 0.385 |
| | | | T5 = 0.085 | |
| | | | T6 = 0.590 | |
| Normalized | NT1 = 0.21 | NT1 = 0.15 | NT1 = 0.09 | NT1 = 0.21 |
| Time | NT2 = 0.26 | NT2 = 0.35 | NT2 = 0.10 | NT2 = 0.22 |
| Parameter | NT3 = 0.07 | NT3 = 0.09 | NT3 = 0.04 | NT3 = 0.07 |
| | NT4 = 0.46 | NT4 = 0.55 | NT4 = 0.10 | NT4 = 0.50 |
| | | | NT5 = 0.08 | |
| | | | NT6 = 0.58 | |
| Pressure | P1 = 34.1 | P1 = 37.5 | P1 = 25.7 | P1 = 34.2 |
| Parameter | P2 = 37.3 | P2 = 40.4 | P2 = 29.6 | P2 = 37.4 |
| (mV) | P3 = 35.1 | P3 = 38.7 | P3 = 28.4 | P3 = 35.2 |
| | P4 = 35.2 | P4 = 38.8 | P4 = 28.5 | P4 = 35.3 |
| | | | P5 = 27.3 | |
| | | | P6 = 27.8 | |
| Normalized | NP1 = 0.914 | NP1 = 0.928 | NP1 = 0.869 | NP1 = 0.914 |
| Pressure | NP2 = 0.941 | NP2 = 0.958 | NP2 = 0.959 | NP2 = 0.942 |
| Parameter | NP3 = 0.944 | NP3 = 0.960 | NP3 = 0.963 | NP3 = 0.945 |
| | | | NP4 = 0.938 | |
| | | | NP5 = 0.940 | |
| Tilting Angle | D1 = 20.65 | D1 = 30.10 | D1 = 43.00 | D1 = 20.06 |
| Parameter | D2 = 11.28 | D2 = 7.39 | D2 = 12.00 | D2 = 13.09 |
| | D3 = 1.82 | D3 = 1.67 | D3 = 2.44 | D3 = 2.00 |
| | D4 = 3.19 | D4 = 3.89 | D4 = 7.50 | D4 = 2.65 |
| | D5 = 31.93 | D5 = 37.49 | D5 = 0.82 | D5 = 33.15 |
| | D6 = 5.01 | D6 = 5.56 | D6 = 3.68 | D6 = 4.65 |
| | | | D7 = 55.00 | |
| | | | D8 = 9.94 | |
| | | | D9 = 4.50 | |
| Normalized | ND1 = 0.415 | ND1 = 0.484 | ND1 = 1.469 | ND1 = 0.411 |
| Tilting Angle | ND2 = 0.227 | ND2 = 0.119 | ND2 = 0.410 | ND2 = 0.268 |
| Parameter | ND3 = 0.037 | ND3 = 0.027 | ND3 = 0.083 | ND3 = 0.040 |
| | ND4 = 0.064 | ND4 = 0.063 | ND4 = 0.256 | ND4 = 0.054 |
| | ND5 = 0.642 | ND5 = 0.603 | ND5 = 0.028 | ND5 = 0.679 |
| | ND6 = 0.101 | ND6 = 0.090 | ND6 = 0.126 | ND6 = 0.094 |
| | | | ND7 = 1.879 | |
| | | | ND8 = 0.339 | |
| | | | ND9 = 0.154 | |
| Area Parameter | A1 = 5.53 | A1 = 3.70 | A1 = 2.49 | A1 = 5.72 |
| | A2 = 7.06 | A2 = 9.10 | A2 = 2.90 | A2 = 5.99 |
| | A3 = 1.10 | A3 = 1.162 | A3 = 1.28 | A3 = 1.94 |
| | A4 = 11.95 | A4 = 13.72 | A4 = 2.81 | A4 = 13.40 |
| | A5 = 12.59 | A5 = 12.80 | A5 = 2.36 | A5 = 11.71 |
| | A6 = 13.05 | A6 = 14.88 | A6 = 15.76 | A6 = 15.34 |
| | A7 = 25.64 | A7 = 27.68 | A7 = 5.39 | A7 = 27.05 |
| | | | A8 = 4.09 | |
| | | | A9 = 18.12 | |
| | | | A10 = 27.60 | |
| Normalized | NA1 = 0.216 | NA1 = 0.134 | NA1 = 0.090 | NA1 = 0.211 |
| Area | NA2 = 0.275 | NA2 = 0.329 | NA2 = 0.105 | NA2 = 0.221 |
| Parameter | NA3 = 0.043 | NA3 = 0.042 | NA3 = 0.046 | NA3 = 0.072 |
| | NA4 = 0.466 | NA4 = 0.496 | NA4 = 0.102 | NA4 = 0.495 |
| | NA5 = 0.491 | NA5 = 0.462 | NA5 = 0.086 | NA5 = 0.433 |
| | NA6 = 0.509 | NA6 = 0.538 | NA6 = 0.571 | NA6 = 0.567 |
| | | | NA7 = 0.195 | |
| | | | NA8 = 0.148 | |
| | | | NA9 = 0.657 | |

Note:
(1) Lower-case a, b, c, d, e, f, g represent time; unit of measurement is sec.
(2) Upper-case A, B, C, D, E, F, G represent the spots on the BP waveform curve; unit of the coordinate axis is (mV, sec.)
(3) Area Parameter (i.e. AabB) is simply defined as a trapezoid area with four angles of points.

What is claimed is:

1. A non-invasive wrist radial artery blood pressure (BP) waveform measuring apparatus system, comprising:
a piezoelectric sensor used for measuring wrist radial artery BP, which can continuously record and generate electric waves on behave of BP pulse waves;
a wristlet with an included air bag, which can be put on one's wrist, and press said piezoelectric sensor;
an air pump capable of pumping up said air bag;
an air escape valve, which is connected with one end of said air bag;
a air duct, which is connected with said air bag;
a circuit module, comprising a central processing unit (CPU), memory, as well as operating software;
a barometer, which is connected with the air duct of said air bag;
a power source, and
a host capable of including the above-mentioned devices.

2. The non-invasive wrist radial artery blood pressure (BP) waveform measuring apparatus system as claimed in claim 1, wherein said piezoelectric sensor can be ceramic Lead piezoelectric zirconate titanate (PZT) piezoelectric transducer, polyvinylidene fluoride (PVDF) piezoelectric transducer, Strain Gauge piezoelectric devices, or Semi-Conductor silicon piezoelectric device.

3. The non-invasive wrist radial artery blood pressure (BP) waveform measuring apparatus system as claimed in claim 1, wherein the main body of said piezoelectric sensor may be a thin film in circular, square, or other geometric shape; its thickness may range between 0.1 mm and 5 mm, and its diameter or width may range between 1 mm and 100 mm.

4. The non-invasive wrist radial artery blood pressure (BP) waveform measuring apparatus system as claimed in claim 1, wherein said piezoelectric sensor is a rectangle thin film, which is installed in the wristlet; wherein one side of said thin film contacts with the air bag in the wristlet, and another side contacts with the outer cloth of said wristlet; when a test is conducted, the wristlet along with the host installed thereon only need to be put on one's wrist to proceed subsequent testing.

5. The non-invasive wrist radial artery blood pressure (BP) waveform measuring apparatus system as claimed in claim 1, wherein a piezoelectric sensing module of said piezoelectric sensor may comprise a number of (at least two) piezoelectric devices, which are disposed in the wristlet; each device is a rectangle (or circular-shaped) piezoelectric thin film, while the length of each side (or diameter) is 3˜5 mm; the base of the sensing module is a soft printed circuit board, which is assembled according to steps as follows: aligning a plurality of piezoelectric devices in one line parallel to hand width, fastening said piezoelectric devices onto a soft printed circuit board, a slot (0.1˜1 mm) is provided between a device and another to prevent the interference of adjacent electric waves.

6. The non-invasive wrist radial artery blood pressure (BP) waveform measuring apparatus system as claimed in claim 1, wherein said circuit module is equipped with a multi-plexer and related driver software for use in receiving electric wave signals from sensing devices, and connected with the filter and magnification circuits.

7. The non-invasive wrist radial artery blood pressure (BP) waveform measuring apparatus system as claimed in claim 1, wherein using the air filling step to obtain optimal testing air pressure, pressurizing the air bag until (or close) this pressure value, and then monitoring the BP waveform.

8. The non-invasive wrist radial artery blood pressure (BP) monitor system as claimed in claim 7, wherein said Optimal testing air pressure (OPAP) represents the maximum air pressure value of primary peak height; under such pressure value, the sensor's BP waveform signal is strongest ever, and its signal-to-noise ratio is highest.

9. The non-invasive wrist radial artery blood pressure (BP) waveform measuring apparatus system as claimed in claim 1, wherein said wristlet may be cloth wristlet with an included air bag; the size of said air bag depends on conventional regulation for measuring one's wrist BP (its length (parallel to hand length) is about 60 to 90 mm, and its width (parallel to hand width) is about 80˜150 mm.

10. The non-invasive wrist radial artery blood pressure (BP) waveform measuring apparatus system as claimed in claim 9, wherein said air bag can be added with a cramp pocket by which the piezoelectric sensing devices can be put therein to press the piezoelectric sensor when the air bag is filled with air.

11. The non-invasive wrist radial artery blood pressure (BP) monitor system as claimed in claim 1, wherein said air escape valve may be an analog electromagnetic valve switch; its size depends on the voltage or current value.

12. The non-invasive wrist radial artery blood pressure (BP) waveform measuring apparatus system as claimed in claim 1, wherein the base of said circuit module is a printed circuit board provided with a central processing unit (CPU), memory (i.e. Flash or RAM), barometer, signal filtering device, signal magnification device, buzzer, real-time clock), and other electric components thereon.

13. The non-invasive wrist radial artery blood pressure (BP) waveform measuring apparatus system as claimed in claim 12, wherein said circuit module may comprise a LCD or LED display; a multi-plexer can be installed to meet prefer circuit requirements.

14. The non-invasive wrist radial artery blood pressure (BP) waveform measuring apparatus system as claimed in claim 1, wherein said circuit module is loaded with a software program.

15. The non-invasive wrist radial artery blood pressure (BP) waveform measuring apparatus system as claimed in claim 13, wherein the operation procedures of said software program include:
controlling the air pressure of said air bag;
searching for Optimal testing air pressure;
measuring radial artery waveform under Optimal testing air pressure;
filtering and magnifying the measured waveform signals;
analyzing and estimating the measured waveform signals.

16. The non-invasive radial artery blood pressure (BP) waveform measuring apparatus system as claimed in claim 1, wherein said power source can be typical dry cells (disposable), a lithium battery (rechargeable), a Ni-NM battery (rechargeable), or other kinds.

17. The non-invasive radial artery blood pressure (BP) waveform measuring apparatus system as claimed in claim 1, wherein said power source can be supplied by conventional alternating current (AC, i.e. 110V or 220V); if alternating current (AC) is used, an AC/DC transformer can be added onto the circuit module to enable devices that only accept the power source for direct current (DC) circuit components.

18. A non-invasive radial artery blood pressure (BP) waveform measuring apparatus system application, wherein the steps of measuring PMBP include:
using the digital BP monitor oscillometric method to measure SBP and DBP;
measuring BP waveforms, and drawing a diagram with X-Y plane (FIG. 7),
wherein Y-axle is relative voltage value V, X-axle is real time t;
measuring the V(mean) of pre-measured BP waveform according to the following formula:
V(mean)=V-t integral value/(t2-t1) (1)
Wherein V(mean) represents relative mean voltage, V-t integral value represents the integral value within t1 (the staiting point of waveform) to t2 (the endpoint of waveform) domain in V-t waveform diagram, t2-t1 represents time interval of individual waveform. PMBP can be measured according to the following formula:
PMBP=V(mean)*(SBP−DBP)/(VS−VD) (2)
Wherein PMBP represents physiological mean blood pressure; V(mean) represents relative mean voltage; SBP represents systolic blood pressure; DBP represents diastolic blood pressure; VS represents the maximum value (peak) of BP waveforms; VD represents the minimum value (trough) of BP waveform;
PMBP of each BP waveform can be estimated according to the following formula; the mean of these PMBP are then estimated according to the following formula:
PMBP(mean)=SUM(PMBP)/N (3)
Wherein PMBP(mean) represents the mean of physiological mean blood pressure (PMBP), SUM(PMBP) represents the sum of N PMBP, N represents the number of BP waveform.

19. A non-invasive radial artery blood pressure (BP) waveform measuring apparatus system application, wherein the steps for identifying testees include:
collecting continuously testees' BP waveform on a regular time (i.e. 50 sec. or 1 min.);
finding out the starting point (trough), endpoint (the starting point of next waveform), and each peak and trough of each BP waveform;
estimating the characteristic parameters of peak number, time, pressure, tilting angle, area, and their normalized characteristic parameters of each BP waveform;
estimating the mean of these parameters within the test period, and defining them as characteristic baseline of the testee's BP waveform;
comparing the testee's BP waveform characteristic parameters with the characteristic baseline described above; if there is a cerain degree of Similarity, the testee can be identified as the same one, otherwise the testee is identified as a different one;
If a group of people (i.e. more than two) undergo measurement of BP waveform characteristic baseline, if one takes the test with unknown identification, the testee's BP waveform characteristic parameters can be compared with others one by one to select one with highest similarity, which can be used to judge if said testee is an identified one; the so-called similarity can be regulated according to experiemtns and identification requirements.

20. The non-invasive radial artery blood pressure (BP) monitor system as claimed in claim 19, wherein personal identification is performed by defining peak number (normally 1.0 to 3.0) of the parameters and normalized time, pressure, tilting angle, and area characteristic parameters as important parameters that satisfy similarity.

21. A non-invasive radial artery blood pressure (BP) waveform measuring apparatus system application, wherein the steps for identifying testees through measuring respiratory waveform and frequency include:
collecting continuously testee's BP waveform within the test period (i.e. 1˜10 min.), and drawing a X-Y diagram, wherein X-value represents time, Y-value represents voltage or pressure;
finding out X value (time value) and Y value (voltage or pressure value) of primary peak point of each waveform;
drawing a continuous XY diagram as the testee's respiratory waveform;
estimating the waveform number (i.e. eight) within a regular period (i.e. one min.) as respirotary frequency or respiratory rate (i.e. eight times per minute).

22. A non-invasive radial artery blood pressure (BP) waveform measuring apparatus system application, wherein the steps for monitoring symptoms of cough or sneeze include:
collecting continuously testees' BP waveform; estimating the mean and standard division of testees' three parameters (that is, primary peak-to-pimary peak time interval, pressure value of primary peak, and pressure value of primary trough) of BP waveform within the initial stage (i.e. one min.), and defining the mean as baseline of the three parameters;
monitoring the three parameters of BP waveform all the time within the test period; if one (or more) of the parameters deviate the baseline and attains predetermined multiple (i.e. triple) of standard division or above, this waveform can be called suspicious irregular data point;
As suspicious irregular data dot occurs randomly (without regular frequency), it can be defined as cough or sneeze;
Cough or sneeze frequency can be obtained (i.e. three times per minute) through statistics.

23. A new physiological signal monitoring apparatus system for uses in hospital quarantine monitoring, comprising:
using the non-invasive accurate BP waveform measuring apparatus system as claimed in claim 1;
using said body temperature measuring technique, and placing temperature sensor into the wristlet;
installing a set of wireless transmission module in the host, and another set into the bedside analyzer in the ward, which can receive patients' physiological signals;
judging if the patient has palpitations, tachypnoea, cough or sneeze in accordance with the HR, BP (SBP, DBP, and PMBP), respiratory waveform and frequency measured through the above-mentioned system;
using said temperature sensor to measure the patient's body temperature from his wrist, and judge if he has favor or not;
using the wireless module to transmit said-physiological signals from the host to bedside analyzer;
using said bedside analyzer to transmit the patient's physiological signals and analysis results out of a ward through Local Area Network (LAN) or the Internet.

24. The new physiological signal monitoring apparatus system for uses in hospital quarantine monitoring as claimed in claim 23, wherein said body temperature measuring technique can employ a small thermocouple, a resistor-type electric device, or infrared optical device; the three devices are thermal-sensitive devices capable of transforming body temperature to voltage or current signals.

25. The new physiological signal monitoring apparatus system for uses in hospital quarantine monitoring as claimed in claim 23, wherein said wireless transmission technique can employ a set of commercial radio frequency (RF) wireless module, its frequency domain may be within frequently-used ISM Band (that is, Industry, Science, Medicine sharing band).

26. An apparatus system for uses in home quarantine monitoring, comprising:
using the non-invasive, accurate BP waveform measuring apparatus system as claimed in claim 1;
using the above-mentioned apparatus system to measure home quarantine person's physiological signals such as HR, BP (SBP, DBP, and PMBP), BP waveform, respiratory waveform and frequency, etc., and adjust if the person has symptoms of palpitations, rapid BP variations, tachypnoea, cough or sneeze accordingly;
using a conventional measuring technique to measure the person's body temperature and adjust if the person has symptoms of favor accordingly;
using conventional wireless transmission techniques to regularly transmit physiological information to a signal processor at home;
using said signal processor to restore, analyze (or reveal) the physiological information, and regularly transmitting the information to related halth organizations through the Internet or Modem;
compiling statistics and regularly summarizing the demographic data of the healthy, sick, and recovered.
